(19) United States
(12) Patent Application Publication
(10) Pub. No.: US 2025/0034638 A1
(43) Pub. Date: Jan. 30, 2025

(54) Articles with Photoiniferter Attached to Inorganic Oxide Surface and Polymers Prepared Therefrom

(71) Applicant: 3M INNOVATIVE PROPERTIES COMPANY, St. Paul, MN (US)

(72) Inventors: Paul B. Armstrong, St. Paul, MN (US); Brylee David B. Tiu, Minneapolis, MN (US); Joshua M. Fishman, St. Paul Park, MN (US)

(21) Appl. No.: 18/704,085

(22) PCT Filed: Nov. 21, 2022
(86) PCT No.: PCT/IB2022/061231

§ 371 Date: Apr. 24, 2024

Related U.S. Application Data
(60) Provisional application No. 63/265,601, filed on Dec. 17, 2021.

(51) Int. Cl. C12Q 1/6874; B01L 3/00

(52) U.S. Cl. CPC C12Q 1/6874; B01L 3/502761; B01L 2200/16; B01L 2300/16

(57) ABSTRACT

Articles (i.e., first articles) with a photoiniferter covalently attached at multiple locations of a substrate having an inorganic oxide coating on a polymeric film are provided. These first articles can be used to form other articles (i.e., second articles) with multiple polymers covalently attached to the inorganic oxide coating of the substrate. The attached polymers are in the form of polymeric brushes extending from the surface of the inorganic oxide coating of the substrate. The polymeric brushes can be functionalized with groups that can bind compounds used in chemical and/or biochemical assays including pharmaceutical assays. The second articles can be used in various analytical devices.

### BACKGROUND

The cost of high-throughput biochemical and/or chemical assays such as nucleic acid and peptide sequencing, or protein, gene, and other chemical and/or biochemical assays is driven by single-use consumables. For example, in addition to chemical and biological reagents used to probe, detect, or measure an unknown sample, the high-throughput assays often require using a patterned flow cell consumable in which the chemical and/or biochemical reagents can selectively react with a target analyte.

Today, patterned flow cells in commercially available devices for the biochemical and/or chemical assays often include glass or silicon substrates with etched wells. The wells in the glass substrates each include chemical functionality selected to bind a chosen target analyte and are separated by regions of an antifouling or non-interacting coatings or surface chemistry. In some examples, the chemical functionality can be directly attached to the substrate via a small molecule linker or can be a functionalized polymer or oligomer, for example a polyacrylamide-containing hydrogel. These patterned flow cells can be manufactured using an intricate and expensive wafer-based photolithographic process that includes multiple chemical-mechanical planarization (CMP), spin coating and washing steps to fill the wells with the correct chemical functionality and place the anti-biofouling coating between the wells. The substrates must then be diced to the correct flow cell size, which also adds costs and requires that the deposited chemistry be protected during this process. Additionally, there are specific shapes of the panels or wafers compatible with the photolithography equipment. This can restrict total flow cell size or lead to portions of the panel or wafer with an incomplete pattern that cannot be used after dicing, for instance if rectangular flow cells are patterned on a circular wafer.

To reduce the financial burden for end users using high-throughput assays and to make the assays more accessible for new markets, it is desirable to reduce the costs of the consumables used in the analysis, including the glass or silicon substrates with etched wells.

### SUMMARY

Articles with a photoiniferter attached at multiple locations of a substrate are provided.

The substrates include a polymeric film with an inorganic oxide coating. The photoiniferter is attached to a surface of the inorganic oxide coating. Polymeric materials can be prepared using these articles. The polymeric materials are in the form of polymeric brushes extending from surface of the inorganic oxide coating. The polymeric brushes can be functionalized with groups that can bind compounds used in chemical and/or biochemical assays, including pharmaceutical assays.

The inorganic oxide coating can cover any portion of the polymeric film in the substrate and is often in the form of a pattern such that the polymeric brushes extend from only a portion of the substrate. For example, the pattern can be in the form of structures (e.g., an array of structures) on the substrate with the polymeric brushes extending from a portion of the structures. Methods of making the article and growing polymeric brushes extending from the inorganic oxide surface of the substrate are also provided.

In a first aspect, a first article is provided. The first article contains (a) a polymeric film, (b) an inorganic oxide coating on at least a first portion of a first surface of the polymeric film, and (c) a plurality of compounds of Formula (I) covalently bonded to the inorganic oxide coating through a condensation reaction with a silyl group (—Si(R¹)ₙ(R²)₃₋ₙ).

Z—(C═S)—S-Q¹-(L)ₚ-[Si(R¹)ₙ(R²)₃₋ₙ]ₘ (I)

In Formula (I), group R¹ is a hydrolyzable group such as an alkoxy, halo, or a group of formula —O—(C═O)—R³ where R³ is a non-hydrolyzable group such as an alkyl. Group R² is a non-hydrolyzable group and is often an alkyl. The variable n is an integer equal to 1, 2, or 3 and the variable m is equal to 1 or 2. The group Z is equal to —N(R⁴)₂, —S—R⁵, —NR⁶R⁷, —OR⁸, or —R⁹. In these formulas for Z, R⁴ is usually an alkyl, aryl, or two R⁴ groups combine to form a 5 or 6 membered ring with nitrogen being one of the ring members and with the ring being saturated or unsaturated; R⁵ is an alkyl or aryl; R⁶ is an alkyl; R⁷ is a nitrogen-containing heteroaryl with 1 or 2 nitrogen ring members; R⁸ is an alkyl or aryl; and group R⁹ is an aryl. Group Q¹ in Formula (I) is equal to —CR¹⁰R¹¹—(C═O)—NH—, —CR¹²R¹³—(C═O)—O—, —C(R¹⁴)(C≡N)—, or —CHR¹⁵—R¹⁶—. In these formulas for Q¹, R¹⁰ is an alkyl; R¹¹ is hydrogen, alkyl, or aryl; R¹² is an alkyl; R¹³ is hydrogen, alkyl, or aryl; R¹⁴ is an alkyl; R¹⁵ is hydrogen or alkyl; and R¹⁶ is an arylene. The variable p is equal to 0 or 1. Group L is present when the variable p is equal to 1 but is absent when the variable p is equal to zero. When present in Formula (I), group L is equal to an alkylene, —R¹⁷—O—(C═O)—NH—R¹⁷—, —R¹⁷—(C═O)—NH—R¹⁷—, or —R¹⁷—S—R¹⁷—, or —N[(R¹⁸)—]₂. In these formulas for group L, each R¹⁷ is independently an alkylene; and R¹⁸ is an alkylene.

In a second aspect, a second article is provided. The second article contains (a) a polymeric film, (b) an inorganic oxide coating on at least a portion of a first surface of the polymeric film, and (c) a plurality of polymeric groups of Formula (II) covalently bonded to the inorganic oxide coating through a condensation reaction with a silyl group (—Si(R¹)ₙ(R²)₃₋ₙ).

Z—(C═S)—S—(POLY)-Q¹-(L)ₚ-[Si(R¹)ₙ(R²)₃₋ₙ]ₘ (II)

The variables n, m, and p as well as the groups R¹, R², L, Q¹, and Z in Formula (II) are defined as in Formula (I). The group POLY is a radically polymerized product of a monomer composition comprising a monomer having an ethylenically unsaturated group.

In a third aspect, a method of making a first article is provided. This method includes providing a polymeric film having a first surface with an inorganic oxide coating on at least a first portion of the first surface of the polymeric film. The method further includes covalently bonding a compound of Formula (I)

Z—(C═S)—S-Q¹-(L)ₚ-[Si(R¹)ₙ(R²)₃₋ₙ]ₘ (I)

to the inorganic oxide coating through a condensation reaction with the silyl group (—Si(R¹)ₙ(R²)₃₋ₙ). The variables n, m, and p as well as the groups R¹, R², L, Q¹, and Z are the same as described above in the first aspect.

In a fourth aspect, a method of making a second article is provided. This method includes forming the first article as described above in the third aspect. The method further includes reacting a first monomer having an ethylenically unsaturated group with the first article in the presence of ultraviolet radiation to form a second article having a compound of Formula (II)

Z—(C═S)—S—(POLY)-Q¹-(L)ₚ-[Si(R¹)ₙ(R²)₃₋ₙ]ₘ (II)

covalently bonded to the inorganic oxide coating through the silyl group —Si(R¹)ₙ(R²)₃₋ₙ. The variables n, m, and p as well as the groups R¹, R², L, Q¹, and Z are the same as described above in the first aspect. The group POLY is a radically polymerized product of a monomer compositions comprising a monomer having an ethylenically unsaturated group.

In a fifth aspect, a device for chemical and/or biochemical analysis is provided that comprises a second article as described above. For example, the device can be used for determination of a DNA sequence.

The terms “a”, “an”, and “the” are used interchangeably and mean one or more.

The term “and/or” means to one or both. For example, the expression X and/or Y means X alone, Y alone, or both X and Y.

The term “azido” refers to a monovalent group —N₃.

The term “alkyl” refers to a monovalent group that is a radical of an alkane and includes groups that are linear, branched, cyclic, bicyclic, or a combination thereof. Unless otherwise indicated, the alkyl groups typically contain from 1 to 20 carbon atoms. In some embodiments, the alkyl groups contain 1 to 10 carbon atoms, 2 to 10 carbon atoms, 1 to 6 carbon atoms, 2 to 6 carbon atoms, 1 to 4 carbon atoms, or 2 to 4 carbon atoms. Cyclic alkyl groups and branched alkyl groups have at least three carbon atoms. Examples of alkyl groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkylene” refers to a divalent group that is a radical of an alkane and includes groups that are linear, branched, cyclic, bicyclic, or a combination thereof. Unless otherwise indicated, the alkylene groups typically contain from 2 to 20 carbon atoms. In some embodiments, the alkylene groups contain 2 to 10 carbon atoms, 2 to 8 carbon atoms, 2 to 6 carbon atoms, 2 to 4 carbon atoms, 3 carbon atoms, or 2 carbon atoms. Cyclic alkylene groups and branched alkylene groups have at least three carbon atoms. Examples of alkylene groups include, but are not limited to, methylene, ethylene, n-propylene, n-butylene, n-pentylene, isobutylene, t-butylene, isopropylene, n-octylene, n-heptylene, ethylhexylene, cyclopentylene, cyclohexylene, cycloheptylene, adamantylene, norbornylene, and the like.

The term “alkoxy” refers to a monovalent group of formula —OR^{a} where Ra is an alkyl as defined above.

The term “aryl” refers to a monovalent group that is a radical of an aromatic carbocyclic compound. The aryl group has at least one aromatic carbocyclic ring and can have 1 to 3 optional rings that are connected to or fused to the aromatic carbocyclic ring. The additional rings can be aromatic, aliphatic, or a combination thereof. The aryl group usually has 5 to 20 carbon atoms or 6 to 10 carbon atoms.

The term “arylene” refers to a divalent group that is a radical of an aromatic carbocyclic compound. The arylene group has at least one aromatic carbocyclic ring and can have 1 to 3 optional rings that are connected to or fused to the aromatic carbocyclic ring. The additional rings can be aromatic, aliphatic, or a combination thereof. The arylene group usually has 5 to 20 carbon atoms or 6 to 10 carbon atoms.

The term “halo” refers to a group that is a chloro, fluoro, bromo, or iodo group.

The term “heteroaryl” refers to a monovalent group that is a radical of an aromatic heterocyclic compound. The heterocyclic ring includes at least one heteroatom selected from nitrogen, sulfur, or oxygen. The heteroaryl group has at least one aromatic heterocyclic ring and can have 1 to 3 optional rings that are connected to or fused to the aromatic heterocyclic ring. The additional rings can be aromatic, aliphatic, or a combination thereof. The heteroaryl group usually has 4 to 20 carbon atoms or 5 to 10 carbon atoms and often has 1 to 3 heteroatoms.

The term “heteroalkylene” refers to alkylene where at least one carbon atom between two other carbon atoms is replaced with a heteroatom. The heteroatom is typically oxygen, nitrogen, or sulfur. The heteroalkylene often has one or more oxygen heteroatoms. The oxygen heteroatoms are not peroxides. In some examples, the heteroalkylene contains multiple ethylene groups or propylene groups separated by oxygen heteroatoms.

The term “(hetero)alkylene” refers to an alkylene, heteroalkylene, or both.

The term “hydrocarbon” refers to refers to a compound or group having only carbon and hydrogen atoms.

The term “hetero-hydrocarbon” refers to a compound or group having carbon, hydrogen, and heteroatoms. Heteroatoms typically include nitrogen, oxygen, and sulfur.

The term “ether group” refers to a group having an oxygen atom between two alkylene groups.

The term “polyether group” is a heteroalkylene having multiple oxygen heteroatoms. The heteroalkylene contains multiple ethylene groups or propylene groups separated by oxygen heteroatoms.

The term “monomeric unit” refers to a polymerized product of a monomer. For example, the monomeric unit associated with the monomer acrylic acid (H₂C═CH—(C═O)—OH) is

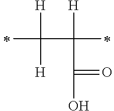

where the asterisk (*) is an attachment site to another monomeric unit or terminal group in the polymer.

The term “thiocarbonylthio” refers to a —S—(C═S)— group.

The term “photoiniferter” is used to refer to a compound or group that can, under appropriate conditions such as exposure to actinic radiation (e.g., ultraviolet radiation), function as a free radical initiator, as a chain transfer agent, and as a free radical chain terminator.

The terms “polymer” and “polymeric material” are used interchangeably and refer to materials formed by reacting one or more monomers. The terms include homopolymers, copolymers, terpolymers, or the like. Likewise, the terms “polymerize” and “polymerizing” refer to the process of making a polymeric material that can be a homopolymer, copolymer, terpolymer, or the like.

The term “click chemistry” refers to the reaction of an unsaturated compound with an azido-containing compound to form a cyclic group having three nitrogen atoms and covalently linking the unsaturated compound to the azido-containing compound. The unsaturated compound typically has a terminal —C≡CH group, a carbon-carbon triple bond in a cyclic group such as a cyclic group with eight ring members, a carbon-carbon double bond in a bicyclic olefinic group such as in norbornene, or an acrylamido group with a carbon-carbon double bond conjugated to a —(C═O)—NH— group. These types of reactions are also referred to as Huisgen reactions.

The terms “in a range of” or “in the range of” are used interchangeably to refer to all values within the range plus the endpoints of the range.

The term “structured film” refers to a film having various structures such as, for example, posts or wells. The dimensions of the structures are often in a range of 50 to 10,000 nanometers or 50 to 1000 nanometers. In some embodiments, the structures can be referred to a “nano-structures” such as “nano-posts” or “nano-wells”. Similarly, the film can be referred to as being “nano-structured”.

Dashes on either side of groups such as —O— and —NH— indicate that these groups are divalent. A dash on a single side of a group such as —(C═O)—OH indicates that this group is monovalent.

### BRIEF DESCRIPTION OF THE DRAWINGS

FIG. 1A is schematic cross-sectional view of a structured film having wells with a photoiniferter bonded to a lower surface of the wells. FIG. 1A is not drawn to scale.

FIG. 1B is schematic cross-sectional view of a structured film having wells with a polymeric material (brush polymers) bonded to a lower surface of the wells. FIG. 1B is not drawn to scale.

FIG. 2A is schematic cross-sectional view of a structured film having posts with a photoiniferter bonded to an upper surface of the posts. FIG. 2A is not drawn to scale.

FIG. 2B is schematic cross-sectional view of a structured film having posts with a polymeric material (brush polymers) bonded to an upper surface of the posts. FIG. 2A is not drawn to scale.

FIG. 3 is a confocal image of a structured film of Example 7 that was functionalized with photoiniferter silane PE1-B, reacted with a monomer composition containing N-(3-aminopropyl) methacrylamide hydrochloride and acrylamide to form brush polymers, and then reacted with a fluorescent dye having an activated ester linkage.

FIG. 4 is a confocal image of a structured film of Example 8 that was functionalized with photoiniferter silane PE2, reacted with a monomer composition containing N-(3-aminopropyl) methacrylamide hydrochloride and acrylamide to form brush polymers, and then reacted with a fluorescent dye having an activated ester linkage.

FIG. 5 is confocal image of a structured film of Example 9 that was functionalized with photoiniferter silane PE3, reacted with a monomer composition containing N-(3-aminopropyl) methacrylamide hydrochloride and acrylamide to form brush polymers, and then reacted with a fluorescent dye having an activated ester linkage.

FIG. 6 is a confocal image of a structured film of Example 11 that was functionalized with photoiniferter silane PE1-B, reacted with a monomer composition containing acrylamide and an azido-containing acrylamide in the presence of a photoiniferter without a silyl group, and then reacted with an alkyne oligonucleotide having a fluorescent group.

FIG. 7 is a confocal image of a structured film of Comparative Example 3 that was functionalized with an acrylamide silane, reacted with a monomer composition containing acrylamide and an azido-containing acrylamide in the presence of a photoiniferter without a silyl group, and then reacted with an alkyne oligonucleotide having a fluorescent group.

FIG. 8 is a confocal image of a structured film of Example 13 that was functionalized with a photoiniferter silane PE1-B, reacted with a monomer mixture containing acrylamide and an azido-containing acrylamide, and then reacted with an alkyne oligonucleotide having a fluorescent group.

### DETAILED DESCRIPTION

Articles (i.e., first articles) with a photoiniferter covalently attached at multiple locations of a substrate having an inorganic oxide coating on a polymeric film are provided. These first articles can be used to form other articles (i.e., second articles) with multiple polymers covalently attached to the inorganic oxide coating of the substrate. The attached polymers are in the form of polymeric brushes extending from the surface of the inorganic oxide coating of the substrate. The polymeric brushes can be functionalized with groups that can bind compounds used in chemical and/or biochemical assays. The second articles can be used in various analytical devices for chemical and/or biochemical assays, including pharmaceutical assays.

### Photoiniferter

The photoiniferter has a silyl group for attachment to the inorganic oxide layer of the substrate. Suitable photoiniferters are of Formula (I).

Z—(C═S)—S-Q¹-(L)ₚ-[Si(R¹)ₙ(R²)₃₋ₙ]ₘ (I)

In Formula (I), group R¹ is a hydrolyzable group such as an alkoxy, halo, or a group of formula —O—(C═O)—R³ where R³ is a non-hydrolyzable group such as an alkyl. Group R² is a non-hydrolyzable group and is often an alkyl. The variable n is an integer equal to 1, 2, or 3 and the variable m is equal to 1 or 2. The group Z is equal to —N(R⁴)₂, —S—R⁵, —NR⁶R⁷, —OR⁸, or —R⁹. In these formulas for Z, R⁴ is usually an alkyl, aryl, or two R⁴ groups combine to form a 5 or 6 membered ring with nitrogen being one of the ring members and with the ring being saturated or unsaturated; R⁵ is an alkyl or aryl; R⁶ is an alkyl; R⁷ is a nitrogen-containing heteroaryl with 1 or 2 nitrogen ring members; R⁸ is an alkyl or aryl; and group R⁹ is an aryl. Group Q¹ in Formula (I) is equal to —CR¹⁰R¹¹—(C═O)—NH—, —CR¹²R¹³—(C═O)—O—, —C(R¹⁴)(C≡N)—, or —CHR⁵—R¹⁶—. In these formulas for Q¹, R¹⁰ is an alkyl; R¹¹ is hydrogen, alkyl, or aryl; R¹² is an alkyl; R¹³ is hydrogen, alkyl, or aryl; R¹⁴ is an alkyl; R¹⁵ is hydrogen or alkyl; and R¹⁶ is an arylene. The variable p is equal to 0 or 1. Group L is present when the variable p is equal to 1 but is absent when the variable p is equal to zero. When present in Formula (I), group L is equal to an alkylene, —R¹⁷—O—(C═O)—NH—R¹⁷—, —R¹⁷—(C═O)—NH—R⁷—, or —R¹⁷—S—R¹⁷—, or —N[(R¹⁸)—]₂. In these formulas for group L, each R¹⁷ is independently an alkylene; and R¹⁸ is an alkylene.

The photoiniferter of Formula (I) has one or two silyl groups of formula Si(R¹)ₙ(R²)₃₋ₙ. It is through a condensation reaction of the silyl group with the inorganic oxide coating on at least a first portion of a first surface of the polymeric film that the photoiniferter is covalently bonded to the substrate. Each silyl group has at least one hydrolyzable group R¹. In some embodiments, there are two or three R¹ groups. The hydrolyzable groups can be an alkoxy, halo, or —O—(C═O)—R³ where R³ is an alkyl. The alkoxy group suitable for R¹ often has 1 to 6, 1 to 4, or 1 to 3 carbon atoms. The halo group suitable for R¹ is often chloro or bromo. When R¹ is equal to —O—(C═O)—R³, the alkyl group R³ often has 1 to 6, 1 to 4, or 1 to 3 carbon atoms. Some example R¹ groups include, but are not limited to, methoxy, ethoxy, acetoxy, and chloro. The R² group is not a hydrolyzable group and is typically an alkyl with 1 to 6, 1 to 4, or 1 to 3 carbon atoms. Some example R² groups include, but are not limited to, methyl, ethyl, and propyl. The variable n is equal to the number of R¹ groups and is an integer equal to 1, 2, or 3. The sum of the R¹ and R² groups is equal to 3 for each silyl group. There can be either one or two silyl groups per photoiniferter (i.e., the variable m is equal to 1 or 2). In many photoiniferters, there is a single silyl group.

When reacted with the inorganic oxide coating of the substrate in the presence of water and/or an alcohol, the R¹ group of the silyl undergoes hydrolysis as well as a condensation reaction with inorganic hydroxy groups (Inorganic-OH) on the surface of the inorganic oxide coating. An example of this reaction is shown schematically in Reaction Scheme A below.

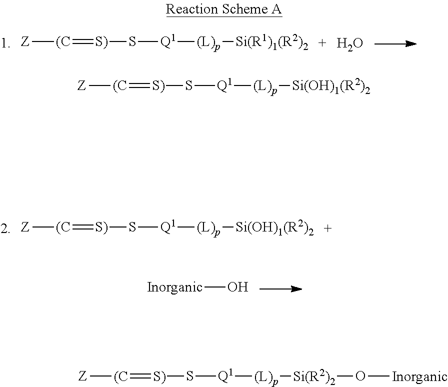

For ease of discussion, the photoiniferter in Reaction Scheme A is shown having a single silyl group (i.e., the variable m in Formula (I) is equal to 1), a single R¹ group (i.e., the variable n is equal to 1), and only one inorganic hydroxide (Inorganic-OH) on the inorganic oxide coating is shown to undergo a condensation reaction with the photoiniferter. However, there are typically multiple inorganic hydroxide groups on the surface of the inorganic oxide coating, there are typically multiple photoiniferter compounds that undergo a condensation reaction with the multiple inorganic hydroxy groups on the surface of the inorganic oxide coating, and there can be more than one R¹ group in a photoiniferter that can react with the inorganic hydroxide groups. The second reaction results in the covalent attachment of a group having a reactive thiocarbonylthio-containing group (—S—(C═S)—Z) to the inorganic oxide coating of the substrate. This reactive thiocarbonylthio-containing groups can be used to initiate and control polymerization of ethylenically unsaturated monomers in the presence of actinic radiation (e.g., ultraviolet radiation) that results in the formation of brush polymers extending from the inorganic oxide surface of the substrate.

The group L in Formula (I) is a linking group between the silyl group and group Q¹, which is a radical leaving group. Group L is optional. When L is absent, the variable p is equal to 0; when L is present, the variable p is equal to 1. In many embodiments, the variable p is equal to 1 and group L is present. Group L is an alkylene, —R¹⁷—O—(C═O)—NH—R¹⁷—, —R¹⁷—(C═O)—NH—R¹⁷—, —R¹⁷—S—R¹⁷—, or —N[(R¹⁸)—]₂. Suitable alkylene L groups often have 1 to 10 carbon atoms such as at least 1, at least 2, or at least 3 and up to 10 or more, up to 8, up to 6, or up to 4 carbon atoms. Some alkylene L groups have 2 to 6 or 2 to 4 carbon atoms. In L groups of formulas —R¹⁷—O—(C═O)—NH—R¹⁷—, —R¹⁷—(C═O)—NH—R¹⁷—, —R¹⁷—S—R¹⁷—, each R¹⁷ is independently an alkylene that often has 1 to 10 carbon atoms such as at least 1, at least 2, or at least 3 and up to 10 or more, up to 8, up to 6, or up to 4 carbon atoms. Some R¹⁷ groups have 2 to 6 carbon atoms or 2 to 4 carbon atoms. In L groups of formula —N[(R¹⁸)—]₂, each R¹⁸ is independently an alkylene having 1 to 10 carbon atoms such as at least 1, at least 2, or at least 3 and up to 10 or more, up to 8, up to 6, or up to 4 carbon atoms and the L group introduces a branding point into the photoiniferter (i.e., the variable m in Formula (I) is equal to 2, which means that there are two silyl groups). Some R¹⁸ groups have 2 to 6 carbon atoms or 2 to 4 carbon atoms.

The L groups are shown within the rest of the photoiniferter in the following Formulas (I-1) to (I-4). Group L is absent in Formula (I-5).

Z—(C═S)—S-Q¹-R¹⁷—O—(C═O)—NH—R¹⁷—Si(R¹)ₙ(R²)₃₋ₙ (I-1)

Z—(C═S)—S-Q¹-R¹⁷—(C═O)—NH—R¹⁷—Si(R¹)ₙ(R²)₃₋ₙ (I-2)

Z—(C═S)—S-Q¹-R¹⁷—S—R¹⁷—Si(R¹)ₙ(R²)₃₋ₙ (I-3)

Z—(C═S)—S-Q¹-N[(R¹⁸)—Si(R¹)ₙ(R²)₃₋ₙ]₂ (I-4)

Z—(C═S)—S-Q¹-Si(R¹)ₙ(R²)₃₋ₙ (I-5)

Some example L groups include, but are not limited to, —CH₂CH₂CH₂—, —CH₂CH₂—(C═O)—NH—CH₂CH₂CH₂—, —CH₂CH₂—O—(C═O)—NH—CH₂CH₂CH₂—, —CH₂CH₂CH₂—S—CH₂CH₂CH₂—, and —N(CH₂CH₂CH₂—)₂. In other examples, L is absent.

The radical leaving group Q¹ in Formula (I) is positioned between the linking group L and the thiocarbonylthio-containing group (—S—(C═S)—Z). Group Q¹ is equal to —CR¹⁰R¹¹—(C═O)—NH—, —CR¹²R¹³—(C═O)—O—, —C(R¹⁴)(C≡N)—, or —CHR¹⁵—R¹⁶—. In these formulas for Q¹, R¹⁰ is an alkyl; R¹¹ is hydrogen, alkyl, or aryl; R¹² is an alkyl; R¹³ is hydrogen, alkyl, or aryl; R¹⁴ is an alkyl; R¹¹ is hydrogen or alkyl; and R¹⁶ is an arylene. Suitable alkyl groups for R¹⁰, R¹¹, R¹², R¹³, R¹⁴, and R¹⁵ each independently has 1 to 10 carbon atoms such as at least 1, at least 2, or at least 3 and up to 10 or more, up to 8, up to 6, or up to 4 carbon atoms. Suitable aryl groups for R¹¹ and R¹³ have 6 to 12 carbon atoms and are often phenyl that can optionally be substituted with an alkyl having 1 to 4 or 1 to 3 carbon atoms. Suitable arylene groups for R¹⁶ have 6 to 12 carbon atoms and are usually phenylene.

The Q¹ groups are shown within the rest of the photoiniferter in the following Formulas (I-5) to (I-8).

Z—(C═S)—S—CR¹⁰R¹¹—(C═O)—NH-(L)ₚ-[Si(R¹)ₙ(R²)₃₋ₙ]ₘ (I-5)

Z—(C═S)—S—CR¹²R¹³—(C═O)—O-(L)ₚ-[Si(R¹)ₙ(R²)₃₋ₙ]ₘ (I-6)

Z—(C═S)—S—C(R¹⁴)(C≡N)-(L)ₚ-[Si(R¹)ₙ(R²)₃₋ₙ]ₘ (I-7)

Z—(C═S)—S—CHR¹⁵—R¹⁶-(L)ₚ-[Si(R¹)ₙ(R²)₃₋ₙ]ₘ (I-8)

Some example Q¹ groups include, but are not limited to, —CH(CH₃)—(C═O)—NH—, —CH(CH₃)—(C═O)—O—, —C(CH₃)₂—(C═O)—NH—, —C(CH₃)₂—(C═O)—O—, —C(CH₃)(Ph)-(C═O)—NH—, —C(CH₃)(Ph)-(C═O)—O—, —C(CH₃)(C≡N)—, —CH₂-Ph- or —CH(CH₃)-Ph- where (Ph) is phenyl and where -Ph- is phenylene.

The group —S—(C═S)—Z is referred to as the thiocarbonyl-containing group and is bonded to the radical leaving group Q¹ in Formula (I). The group Z is equal to —N(R⁴)₂, —S—R⁵, —NR⁶R⁷, —OR⁸, or —R⁹. In these formulas for Z, R⁴ is an alkyl, aryl, or two R⁴ groups combine to form a 5 or 6 membered ring with nitrogen being one of the ring members and with the ring being saturated or unsaturated; R⁵ is an alkyl or aryl; R⁶ is an alkyl; R⁷ is a nitrogen-containing heteroaryl with 1 or 2 nitrogen ring members; and R⁸ is an alkyl or aryl; and group R⁹ is an aryl. Suitable alkyl groups for R⁴, R⁵, R⁶, and R⁸ typically have 1 to 10 carbon atoms such as at least 1, at least 2, or at least 3 and up to 10 or more, up to 8, up to 6, or up to 4 carbon atoms. Suitable aryl groups for R⁴, R⁵, R⁸, and R⁹ often have 6 to 12 carbon atoms and are often phenyl that can optionally be substituted with an alkyl having 1 to 4 or 1 to 3 carbon atoms. Suitable R⁷ groups typically have heteroaryl groups with 5 or 6 ring members.

The —S—(C═S)—Z groups are shown within the rest of the photoiniferter in the following Formulas (I-9) to (I-13).

(R⁴)₂N—(C═S)—S-Q¹-(L)ₚ-[Si(R¹)ₙ(R²)₃₋ₙ]ₘ (I-9)

R⁵—S—(C═S)—S-Q¹-(L)ₚ-[Si(R¹)ₙ(R²)₃₋ₙ]ₘ (I-10)

R⁷R⁶N—(C═S)—S-Q¹-(L)ₚ-[Si(R¹)ₙ(R²)₃₋ₙ]ₘ (I-11)

R⁸O—(C═S)—S-Q¹-(L)ₚ-[Si(R¹)ₙ(R²)₃₋ₙ]ₘ (I-12)

R⁹—(C═S)—S-Q¹-(L)ₚ-[Si(R¹)ₙ(R²)₃₋ₙ]ₘ (I-13)

Some example groups of formula —S—(C═S)—Z include, but are not limited to, those of formula —S—(C═S)—N(R⁴)₂ where each R⁴ is independently an alkyl having 1 to 4 carbon atoms, a phenyl, or an unsaturated five-membered ring with a nitrogen being one of the ring members, those of formula —S—(C═S)—S—R⁵ where R⁵ is an alkyl having 1 to 4 carbon atoms or phenyl, those of formula —S—(C═S)—NR⁶R⁷ where R⁶ is methyl and R⁷ is a six-membered heteroaryl with a nitrogen ring-member, and those of formula —S—(C═S)—OR⁸ where R⁸ is an alkyl having 1 to 4 carbon atoms or a phenyl.

Photoiniferters of Formula (I) include, but are not limited to, the following specific compounds (I-A) to (I-I).

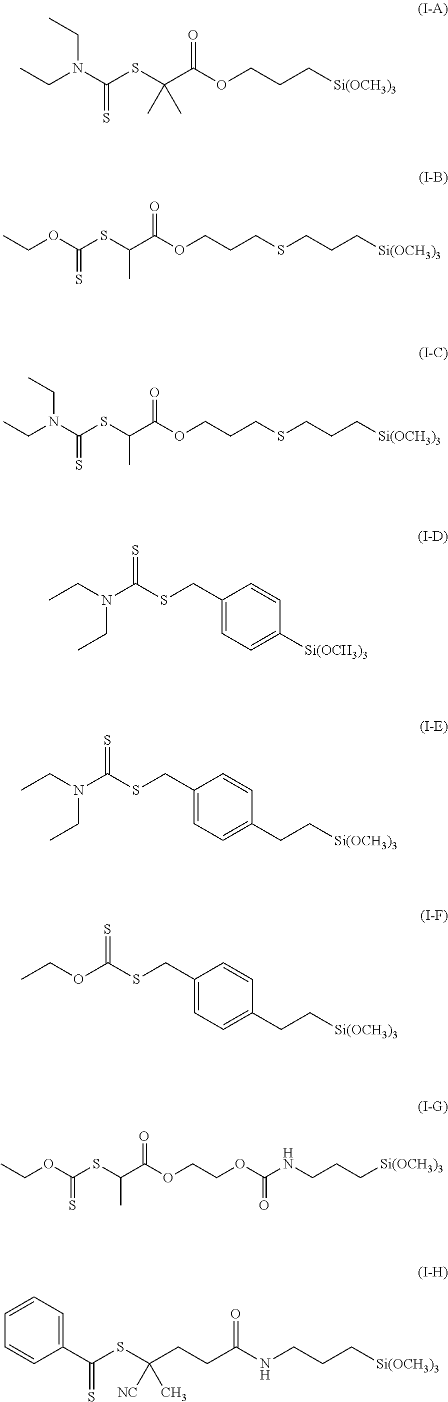

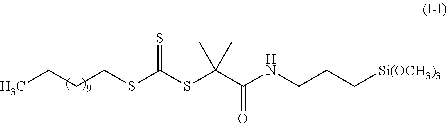

### First Article: Photoiniferter Attached to Substrate

A first article is provided that includes a photoiniferter of Formula (I) covalently attached to a substrate. The substrate contains a polymeric film and an inorganic oxide coating on at least a portion of an outer surface (i.e., first surface) of the polymeric film. The photoiniferter is covalently attached to the inorganic oxide coating.

More specifically, the polymeric film of the substrate has a first surface and a second surface opposite the first outer surface. The inorganic oxide coating is adjacent to a first portion of the first surface of the polymeric film. The first portion is typically in a range of 10 to 100 percent of the first surface of the polymeric film. For example, the first portion can be at least 10, at least 20, at least 30, at least 40, at least 50, or at least 60 percent and up to 100, up to 90, up to 80, up to 70, up to 60, up to 50, or up to 40 percent of the first surface of the polymeric film. In the portion of the first surface of the polymeric film that is not adjacent to the inorganic oxide coating, there can be either no coating or a coating that is different than the inorganic oxide coating. This other portion of the first surface of the polymeric film that does not have an adjacent inorganic oxide coating can be referred to herein as the second portion. The photoiniferter is covalently bonded to the inorganic oxide coating that is adjacent to the first portion of the polymeric film but is not covalently bonded to the second portion of the polymeric film that lacks an inorganic oxide coating.

The substrate can be planar such as in the form of a flat sheet or can contain a plurality of structures. The structures can be of any desired size and shape. The structures can be formed either prior to or after placement of the inorganic oxide coating adjacent to the first portion of the first surface of the polymeric film.

The polymeric film can be of any desired composition. For use in some applications such as those directed to biochemical and/or chemical assay, various fluorescent materials are used during the analysis. Because of this, it is often preferable that the polymeric film have low autofluorescence to minimize the contribution of the polymeric film to the fluorescent signals detected during analysis. The autofluorescence of the polymeric film can be controlled, for example, by varying the thickness and composition of the polymeric film. Preferably, the polymeric film has an autofluorescence measured in a wavelength range of 400 to 800 nanometers (nm) that is close to or not significantly greater than that of borosilicate glass or other substrates commonly used in biochemical and/or chemical assays.

Examples of low autofluorescence films include, but are not limited to, polyolefins such as cyclic olefin polymers or copolymers, biaxially oriented polypropylene, poly(meth)acrylates, polyamides, polyesters, polycarbonates, hydrogenated styrene, and combinations thereof.

Polymeric material with a higher autofluorescence can be used if the thickness of the film is suitably low. The total thickness of the polymeric film is often in a range of 15 to 1000 micrometers. For example, the thickness can be at least 15, at least 20, at least 25, at least 30, at least 50, at least 75, at least 100, at least 200, or at least 500 micrometers and up to 1000, up to 750, up to 500, up to 200, up to 100, up to 75, or up to 50 micrometers.

The polymeric film can include a single layer of polymeric material or can contain multiple layers of polymeric material. For example, often the polymeric film has a first layer that is a support film and then one or more additional layers on which the inorganic oxide layer is formed. The additional layers can include various structures.

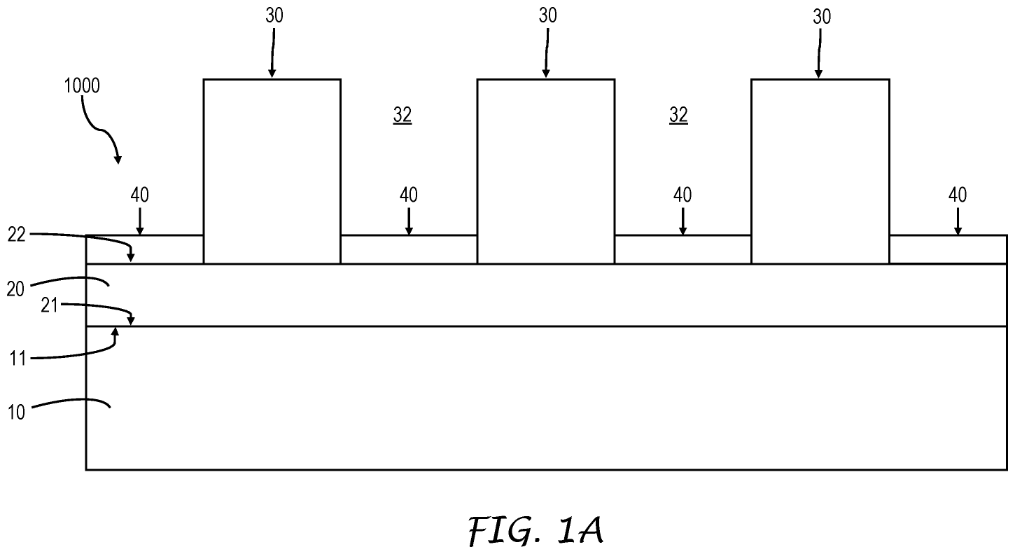

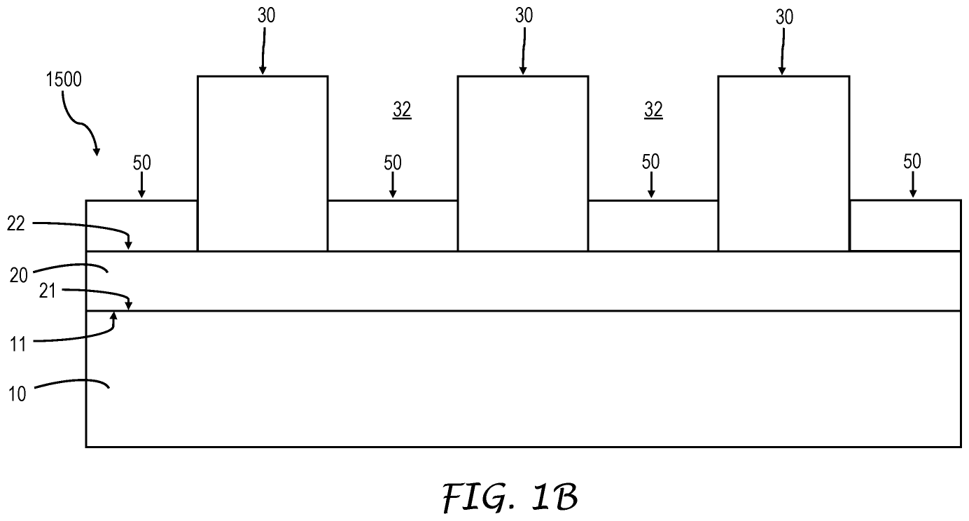

A first embodiment of the first article is shown in FIG. 1A. The first article 1000 includes the polymeric film 10 having a first surface 11 that is adjacent to the inorganic oxide coating 20. The inorganic oxide coating 20 has a first surface 21 that is adjacent to the first surface 11 of the polymeric film 10 and a second surface 22 that is opposite the first surface 21 of the inorganic oxide coating 20. Adjacent to the second surface 22 of the inorganic oxide coating 20 are a plurality of structures 30 extending away from the second surface 22 of the inorganic oxide coating 20. The plurality of structures 30 can have any desired shape and any desired size. The structures 30 are interspersed with land areas 32. The land areas 32 includes a portion of the second surface 22 of the inorganic oxide coating that is available for binding to photoiniferter compounds of Formula (I).

Although the ratio of the area (or length in one dimension) of the land areas 32 to that of the structures 30 are shown as being about 1:1 in FIG. 1, any suitable ratio can be used. As used herein, the width or area refers to the distance or area of the second surface 22 of the inorganic oxide layer adjacent to the structures 30 and the land areas 32. The ratio can be for example in a range of 100:1 to 1:100, 50:1 to 1:50, 20:1 to 1:20, 10:1 to 1:10, 5:1 to 1:5, or 2:1 to 1:2.

The inorganic oxide coating 20 can be formed in any desired manner and can contain any suitable inorganic oxide. The inorganic oxide is typically selected so that there are sufficient inorganic hydroxy groups on the second surface 22 of the inorganic oxide coating 20 to undergo a condensation reaction with the silyl group of the photoiniferter of Formula (I). In many embodiments, the inorganic oxide coating contains various silicon oxides such as silicon oxides, silicon carbon oxides, silicon aluminum oxides, titanium oxides, aluminum oxides, tin oxides, germanium oxides, gallium oxides, zinc oxides, indium oxides, and mixtures or combinations thereof. Mixtures of a plurality of different types of inorganic oxides can be used. In many embodiments, the inorganic coating is predominately silicon oxides, silicon aluminum oxides, silicon carbon oxides, and mixtures or combinations thereof. The inorganic oxide can be deposited on the first surface 11 of the polymeric film 10 using a technique such as sputtering, plasma-enhanced chemical vapor deposition (PECVD), or atomic layer deposition (ALD). The thickness of the inorganic oxide coating is often in a range of 1 to 500 nm. The thickness can be up to 500 nm, up to 400, up to 300, up to 200, up to 100, or up to 50 nm and at least 1 nm, at least 2 nm, at least 5 nm, at least 10 nm, at least 20 nm, at least 25 nm, at least 40 nm, or at least 50 nm.

The structures 30 can be arranged on the second surface 22 of the inorganic oxide coating in a regular or irregular array. As shown in FIG. 1A, the structures 30 are cylindrical or cubical columns or posts but the structures can have other shapes such as spherical, hemispherical, rectangular, pyramidal, trapezoidal, and the like.

The structures 30 can be formed by any known process such as, for example, photolithography, micro-contact or inkjet printing, nanoimprint lithography, laser patterning, and combinations thereof. The structures can have any desired size and can be arranged in any desired pattern. In some embodiments, the plurality of structures 30 include a regular array of cylindrical, cubic, or rectangular posts with a diameter ranging from about 50 nm to about 10,000 nm (i.e., 10 micrometers). For example, the diameter can be at least at least 50, at least 100, at least 200, at least 300, at least 400, or at least 500 nm and up to 10,000, up to 7500, up to 5000, up to 2000, up to 1500, up to 1000, up to 800, up to 600, or up to 500 nm. The aspect ratio (height:diameter) is often in a range of 5:1 to 1:70. This ratio can be up to 5:1, up to 4:1, up to 2:1, up to 1:1 and at least 1:70, at least 1:50, at least 1:20, at least 1:10, at least 1:1, or at least 1:1. Typical structures 30 are designed to separate the land area 32 by enough space to resolve fluorescently labeled molecules attached to the surface in the land area 32 using optical microscopy. In some cases, the inorganic oxide is roughened to improve the adhesion to structures 30.

The structures 30 are typically formed of a material that will not bind either the photoiniferter or various materials used in biochemical and/or chemical assays. For example, the structures 30 are often formed from anti-biofouling materials that resist or prevent accumulation of biological species such as, for example, microorganisms, nucleic acids, amino acids, DNA fragments, proteins, target analytes, sequencing reagents, and fluorophores.

Example anti-biofouling materials suitable for forming the structures 30 include, but are not limited to, noble metals (e.g., gold, platinum, and alloys thereof that do not have sufficient inorganic hydroxy groups on an outer surface for binding to other materials), fluoropolymers, or various materials having a hydrocarbon surface. Example fluoropolymers that can be used include, but are not limited to, those commercially available under the trade designation CYTOP from AGC Chemicals (Exton, PA, USA), various terpolymers of tetrafluoroethylene, hexafluoropropylene, and vinylidene fluoride (THV) commercially available from 3M Dyneon (Saint Paul, MN, USA), and various materials commercially available under the trade designation LTM from Solvay (Alpharetta, GA, USA). Examples of materials having a hydrocarbon surface can be formed using several known methods. A first method includes forming a methyl terminated surface by molecular fragmentation of a compound such as hexamethyldisiloxane (HMDS) through plasma dissociation. Alkyl terminated surfaces can also be formed by plasma enhanced chemical vapor deposition of materials such as tetraethyl orthosilicate, tetramethyl silane, hexamethyldisilane, or trimethylamine. Alternatively, the alkyl terminated surfaces can be formed from the polymeric products of various silicone-containing acrylates or polydimethylsiloxanes.

The structures 30 do not cover the entire second surface 22 of the inorganic oxide coating.

In some embodiments, the land area 32 between the structures 30 can function as wells. The land area 32 has an exposed second surface 22 of the inorganic oxide coating and the photoiniferter of Formula (I) can be attached to the substrate in these locations. Multiple photoiniferter compounds can be attached to surface 22 of the inorganic oxide coating in the land area 32 (i.e., wells). That is, the multiple attached photoiniferters can form a photoiniferter layer 40. The reactions involved in attachment of the photoiniferter to the inorganic oxide coating are illustrated in Reaction Scheme A above. Brush polymers can be grown from the photoiniferter layer 40 containing multiple attached photoiniferters to form second articles. As shown in FIG. 1A, the photoiniferter layer 40 can be viewed as being wells with a width in a range from 50 nm to about 10,000 nm or from 50 to 1000 nm and can be referred to as “nano-wells”. For example, the width can be at least at least 50, at least 100, at least 200, at least 300, at least 400, or at least 500 nm and up to 10,000, up to 7500, up to 5000, up to 2000, up to 1500, up to 1000, up to 800, up to 600, or up to 500 nm.

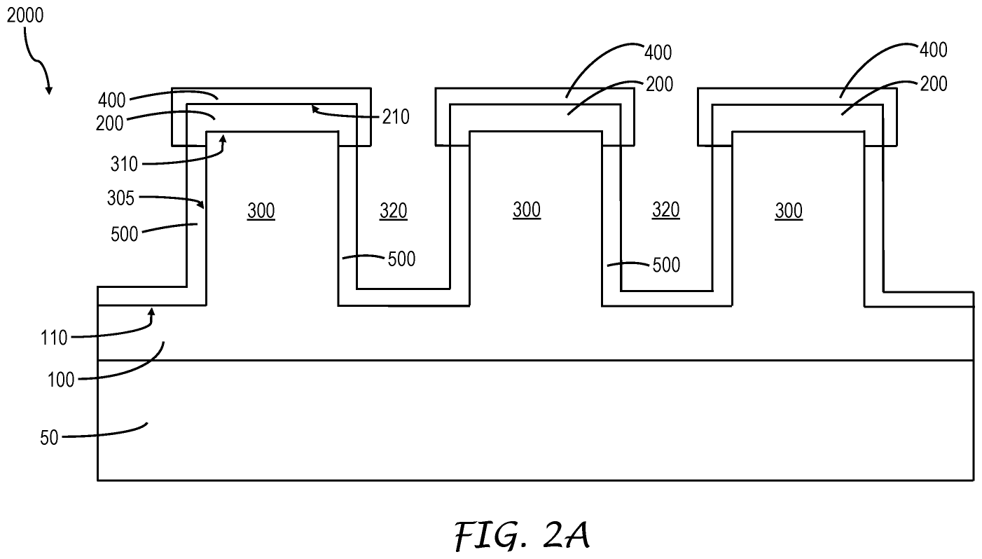

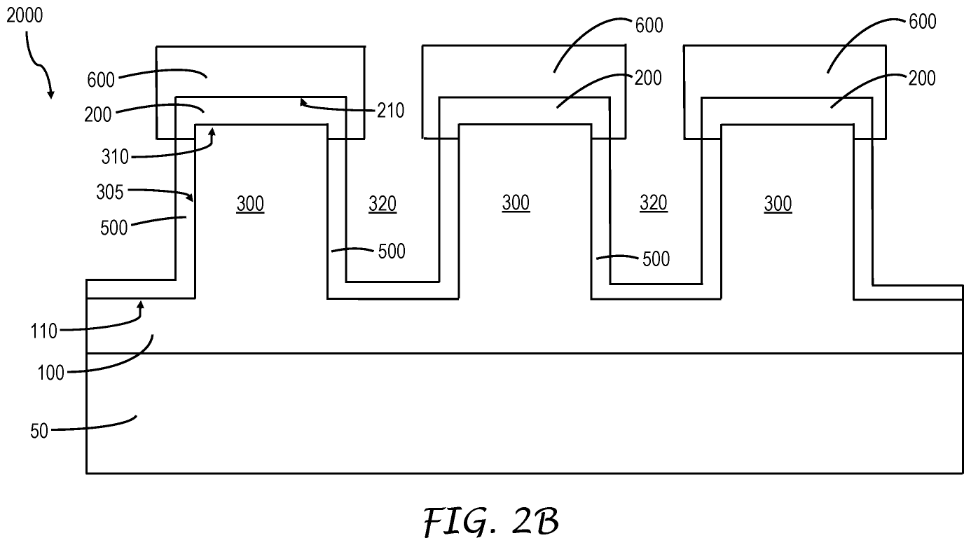

A second embodiment of the first article is shown in FIG. 2A. The first article 2000 includes a two-layer polymeric film. The first layer is a support film 50 having dimensions typically ranging from about 25 to 1000 micrometers in thickness and a second polymeric film 100 that is not a flat sheet but that has structures 300 on a first surface 110 of the polymeric film. Although the thickness of the second polymeric film can vary across the article, the thickness is typically no greater than 1 micrometer including the structures 300. The thickness can be, for example, 0.1 micron or less, at least 0.1 micrometers, at least 0.2 micrometers, at least 0.3 micrometers, or at least 0.5 micrometers and up to 1 micrometer, up to 0.8 micrometers, up to 0.6 micrometers, or up to 0.5 micrometers. The structures 300 can be formed, for example, by microreplication against a structured tool, casting, and other known methods. The size and shape of the structures 300 are like those of structures 30 in FIG. 1A. Between the structures 300, there is plurality of land areas 320. The size and shape of the land areas can be similar to those of land areas 32 in FIG. 1A. The land area 320 has a first surface 110 this is below the upper surface 310 of the structures 300. Typical land area 320 are designed to separate structures 300 by enough space to resolve fluorescently labeled molecules attached to the surface of 300 using optical microscopy. The structures 300 often have a width in a range of 50 nm to about 10,000 nm or from 50 to 1000 nm and can be referred to as “nano-posts”.

In this second embodiment of the first article, there is an anti-biofouling coating 500 adjacent to a first surface 110 of the land area 320. Additionally, there is an anti-biofouling coating 500 adjacent to at least a portion the sides 305 of the structures 300. The anti-biofouling coating is not present on (or has been removed from) the upper surfaces 310 of the structures 300. Although FIG. 2A (as well as FIG. 2B) shows only a relatively narrow layer 200 compared to thickness (i.e., height) of the structures 300, the ratio of the thickness of the layer 200 to layer 300 can be any desired value. FIG. 2A (as well as FIG. 2B) shows layer 200 on top of structures 300 and extending partially down the side 305. Layer 200 will extend to meet where anti-biofouling coating 500 ends on the side wall 305. The interface between layer 200 and 500 can occur anywhere on wall 305 as long as an unbroken layer of 500 remains on the land area between the posts. It is also possible that layer 500 extends to the top of side 305 and layer 200 is only located on the top 310.

The antifouling coating 500 is often formed from the same types of hydrocarbon coatings described for use to prepare the structures 30 in FIG. 1A. The thickness of the anti-fouling coating is often in a range of 2 to 500 nm. For example, the thickness can be at 2, at least 3, at least 5, at least 10, at least 20, or at least 50 nm and up to 500, up to 400, up to 300, up to 200, up to 100, or up to 50 nm.

Rather than having the inorganic oxide coating positioned on the land surfaces 22 as in FIG. 1A, the inorganic oxide coating 200 is positioned on the upper surfaces 310 of the structures 300 that are part of the polymeric film as shown in FIG. 2A. That is, the inorganic oxide coatings 200 are on the top of the posts (structures 300) rather than on surface 110 of the land area 320. Stated differently, FIG. 1A shows an embodiment where the inorganic oxide coating is on a bottom surface of a plurality of wells while FIG. 2A shows an embodiment where the inorganic oxide coating is on an upper surface of a plurality of posts. The thickness and composition of the inorganic oxide coating 200 in FIG. 2A can be the same as the thickness and composition of the inorganic oxide coating 20 in FIG. 1A.

Although the ratio of the width of the land areas 320 to that of the structures 300 are shown as being about 1:1 in FIG. 2A, any suitable ratio can be used. The ratio can be for example in a range of 100:1 to 1:100, 50:1 to 1:50, 20:1 to 1:20, 10:1 to 1:10, 5:1 to 1:5, or 2:1 to 1:2.

In FIG. 2A, multiple photoiniferter compounds 400 are covalently attached to the outer surface 210 of the inorganic oxide coating 200. The reactions involved in attachment of the photoiniferter compounds to the inorganic oxide coating are illustrated in Reaction Scheme A above. Brush polymers can be grown from the attached photoiniferter to form second articles as described below. The multiple photoiniferters can be viewed as being covalently attached to a plurality of posts in FIG. 2A.

The first article can further include multiple polymeric film layers. For example, there can be a support film adjacent to the surface of the polymeric film 10 opposite the first surface 11 in FIG. 1A or adjacent to the polymeric film 100 opposite the first surface 110 in FIG. 2A. The support film 50 is shown in FIGS. 2A and 2B. Like the polymeric film 10 and 100, the support film is typically selected to have a low autofluorescence. The support film can be considered as a second layer of the polymeric film. An adhesive may optionally be present between support film 10 and polymeric film 100 or there may be an adhesive below support film 50.

Any suitable method of making the first articles of FIG. 1A and FIG. 2A can be used. Advantageously, the first articles can be prepared in a continuous manufacturing process, which can provide higher throughput and lower manufacturing costs compared to water-based photolithographic process methods that are generated on a part-by-part basis. Continuous manufacturing processes, which in some cases can be referred to as a roll-to-roll processes, also provide several advantages and increased design flexibility relative to silicon wafer or glass panel processing techniques when producing a structured substrate. For example, when structured substrates are made using a silicon wafer or glass panel, grafting analyte binding chemistry on post-like structures extending away from the surface of the wafer can be difficult; as a result, in silicon wafer or glass panel constructions the binding chemistry can be limited to the depressed well-like areas of the wafer. In addition, due to the necessary flexibility of the polymer carrier web, the inorganic layer can be thin when roll-to-roll processing is used. For example, the inorganic layer can be less than 200 nm, if desired. Further, in contrast to traditional wafer processing, amorphous silicon oxide layers deposited by roll-to-roll processing may include impurities such as aluminum or carbon to allow efficient deposition rates on flexible, temperature sensitive surfaces using processing techniques such as sputtering, atomic layer deposition (ALD) or plasma enhanced chemical vapor deposition (PECVD). Furthermore, converting patterns produced on films to the correct part size is a less expensive and labor-intensive process then dicing wafers or panels.

One method for preparing the first article involves the formation of wells with the photoiniferter covalently attached to the bottom of the wells as shown in FIG. 1A. A support film having low autofluorescence is provided. This support film is then coated with an inorganic oxide such as silicon carbon oxide or silicon aluminum oxide using a method such as plasma enhanced chemical vapor deposition (PECVD). Optionally this oxide layer can have random nanotexture created by depositing a silicon containing discontinuous layer using PECVD while either simultaneously or sequentially etching the surface with a reactive species as described in U.S. Pat. Nos. 10,134,566 and 8,634,146 of David et al. This random nanotexture can enhance the adhesion of the subsequent layer. Additionally, a coupling agent such as an aminosilane can be applied to the inorganic oxide to further enhance adhesion of the subsequent layer. For example, the aminosilane can be bis(trimethoxysilylpropyl)amine (available as SILQUEST A-1170 from Momentive (Waterford, NY, USA)).

Next, an anti-biofouling layer is coated on the inorganic oxide. This layer can be a perfluorinated polymer such as THV, which is a terpolymer of tetrafluoroethylene, hexafluoropropylene, and vinylidene fluoride. This layer often has a thickness of about 10 to 100 nm. Optionally, a tie layer can then be coated on the antibiofouling layer. For example, if the anti-biofouling layer is a perfluorinated polymer, the tie layer can be a thin layer of poly(allyl amine) (having a thickness of 10 nm to about 100 nm) which will bind to subsequently applied acrylates. In another embodiment, the tie layer can be a washable layer such as poly(vinyl alcohol). The optional tie layer often has a thickness of about 10 to 100 nm.

The anti-fouling layer is then overcoated with a UV-curable replication resin to create a patterned surface with wells such as nano-wells. The UV-curable replication resin is pressed against a structured nickel surface attached to a roller. This resin composition often contains a (meth)acylate polymer or copolymer. For example, the resin layer can be formed from a resin composition containing an oligomer (e.g., a urethane (meth)acylate oligomer), one or more crosslinkers having a plurality of (meth)acryloyl groups (e.g., 1,6-hexanediol diacrylate (HDDA) and/or trimethylolpropane triacrylate (TMPTA)), and a photoinitiator. The thickness of the resin layer is selected so that the structured nickel surface is fully wetted. The resin layer is exposed to UV radiation through the support film while in contact with the structured nickel surface. After peeling the partially cured resin layer from the structured nickel surface, the outer surface of the resin layer is further exposed to UV radiation for further curing. This results in a pattern of wells (e.g., nano-wells) over the anti-biofouling layer.

The pattern of wells (e.g., nano-wells) is then transferred from the patterned surface to the anti-biofouling layer using an etching step such as, for example, reactive ion etching using a fluorine compound. The depth of the etch is controlled based on the duration and selectivity of the etch to expose inorganic oxide in the bottom of the wells but leave behind some replication resin in the regions above the wells. This residual resin protects the anti-biofouling layer from oxidative damage.

Next, the residual replication resin and optional tie layer is removed by peeling to expose the anti-biofouling layer. More specifically, the etched film is covered with a curable composition that contains one or more monomers having a plurality of (meth)acylate groups. In many embodiments, all or the majority of the monomers in the curable composition have a plurality of (meth)acrylate groups. The curable composition is then covered with another film such as a layer of MELINEX film (e.g., MELINEX 454), which is a polyester (PET) film available from TEKRA (New Berlin, WI). The curable composition is cured with UV radiation. When the cured composition on the MELINEX film is peeled away, the replication resin above the wells (e.g., nano-wells) is removed. That is, the remaining replication resin is transferred to the MELINEX film. This resulting film with wells (e.g., nano-wells) can then be reacted with a photoiniferter having a silyl group of Formula (I) to covalently attached the photoiniferter to the wells.

Another method for preparing the first article of FIG. 2A is described further in the Example section below. This results in the formation of posts with the photoiniferter covalently attached to the top of the posts. A support film having low autofluorescence is provided. The support film can be, for example, a polyester film such as polyethylene terephthalate. This support film can optionally be overcoated with an optional primer resin such as, for example, those commercially available under the trade designation RHOPLEX (e.g., RHOPLEX 3208) from Dow Chemical (Midland, MI) that contains a 4:1 ratio of acrylic to melamine-formaldehyde curing resin. Another resin layer is applied over the support layer (and optional primer layer). The resin layer is formed from a UV-curable replication resin that contains a (meth)acylate polymer or copolymer. For example, the resin layer can be formed from a resin composition containing an oligomer (e.g., a urethane (meth)acylate oligomer), one or more crosslinkers having a plurality of (meth)acryloyl groups (e.g., 1,6-hexanediol diacrylate (HDDA) and/or trimethylolpropane triacrylate (TMPTA)), and a photoinitiator.

The support film coated with the resin layer is pressed against a structured nickel surface attached to a roller. The thickness of the resin is selected so that the structured nickel surface is fully wetted. The resin layer is exposed to UV radiation through the support film while in contact with the structured nickel surface. After peeling the partially cured resin layer from the structured nickel surface, the outer surface of the resin layer is further exposed to UV radiation for further curing. This process results in the formation of the polymeric film 100 having structures 300 and land areas 320 shown in FIG. 2A. The optional support film is not shown in FIG. 2A.

Next, the anti-biofouling layer or release layer is deposited on all outer surfaces of the polymeric film. That is, both the structures 300 and the land areas 320 are covered with the anti-biofouling layer. The anti-biofouling layer can be formed by plasma enhanced chemical vapor deposition of hexamethyldisiloxane (HMDSO). The deposited layer typically has a methylated surface.

A planarizing layer is then formed to cover all the deposited anti-fouling layer (release layer) to fill in the land areas 320 as well as to cover the structures 300. The planarizing layer can be formed, for example, using a roll-to-roll process and die coating a polymeric solution (e.g., a solution of polyvinyl butyral dissolved in isopropanol). After deposition, the coating is dried to create the planarizing layer.

The planarized layer is then subjected to reactive ion etching. Using plasma enhanced chemical vapor deposition methodology in the presence of oxygen gas, the planarizing layer is removed until the underlying release layer on the top of the posts is exposed. The oxygen exposure oxidizes the HMDSO on the top of the structures 300 resulting in the formation of an inorganic oxide layer. Once the tops of the posts are exposed, the etching process is terminated. The etched tops of the posts contain various silicon carbon oxides.

The remaining planarizing layer that is within the land areas 320 is removed by peeling. More specifically, the etched film is covered with a curable composition that contains one or more monomers having a plurality of (meth)acylate groups. In many embodiments, all or the majority of the monomers in the curable composition have a plurality of (meth)acrylate groups. The curable composition is then covered with another film such as a layer of MELINEX film (e.g., MELINEX 454), which is a polyester (PET) film available from TEKRA (New Berlin, WI). The curable composition is cured with UV radiation. When the cured composition on the MELINEX film is peeled away, the polyvinyl butyral in the land areas 320 are removed. That is, the remaining polyvinyl butyral is transferred to the MELINEX film. This product can then be reacted with a photoiniferter compound of Formula (I) to form the structure illustrated in FIG. 2A.

### Second Article: Brush Polymers Attached to Substrate

A second article is provided that includes a plurality of brush polymers covalently attached to a substrate. The second article is prepared from the first article described above. More specifically, the first article, which has a plurality of covalently attached photoiniferters, is reacted with various monomers in the presence of ultraviolet radiation to grow a plurality of brush polymers extending from and covalently attached to the inorganic oxide surface of the substrate. These reactions are illustrated in Reaction Scheme B.

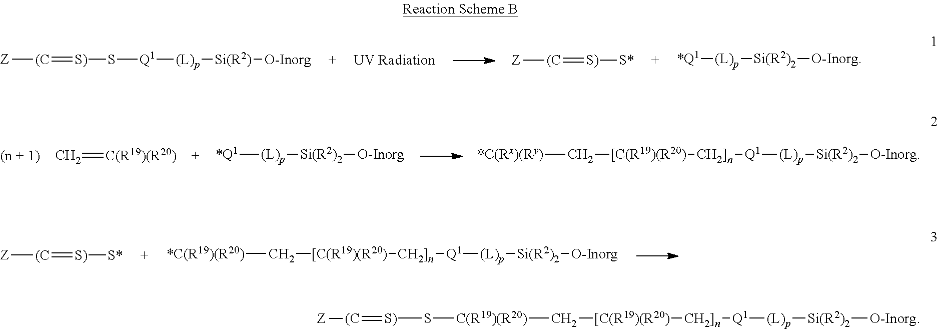

In a first reaction step, exposure of the first article (Z—(C═S)—S-Q¹-(L)ₚ-Si(R²)₂—O-Inorg) to actinic radiation (e.g., ultraviolet radiation) results in formation of both a radical of first article (*Q¹-(L))ₚ Si(R²)₂—O-Inorg) and a radical of the thiocarbonylthio-containing group (*S—(C═S)—Z). A first monomer having an ethylenically unsaturated group (shown for simplicity as CH₂═CR¹⁹R²⁰ in Reaction Scheme B where R¹⁹ and R²⁰ are any suitable groups on an ethylenically unsaturated monomer) reacts with a radical *Q¹-(L)ₚ-Si(R²)₂—O-Inorg resulting in the generation of a second radical that can react with another monomer. This process may continue until all the monomer has reacted. The polymerization of the (n+1) moles of the first monomer is shown as the product of this reaction, which is *C(R^{x})(R^{y})—CH₂—[C(R^{x})(R^{y})—CH₂]ₙ-Q¹-(L)ₚ-Si(R²)₂—O-Inorg. At any point in this process, the growing radical may recombine with the thiocarbonylthio radical (*S—(C═S)—Z) as shown in Reaction 3 to form Z—(C═S)—S—C(R^{x})(R^{y})—CH₂—[C(R^{x})(R^{y})—CH₂]ₙQ¹-(L)ₚ-Si(R²)₂—O-Inorg. Upon continued exposure to actinic radiation, the radicals Z—(C═S)—S* and *C(R^{x})(R^{y})—CH₂—[C(R^{x})(R^{y})—CH₂]n-Q¹-(L)ₚ-Si(R²)₂—O-Inorg can form from the product of Reaction 3. If more monomers are present or added, the regenerated radical can undergo further polymerization. Eventually, this radical will combine again with a thiocarbonylthio radical. The polymerization reaction stops when exposure to actinic radiation is stopped. The product is a second article having a plurality of polymeric chains (e.g., brush polymers) grafted to the surface inorganic oxide coating. At least some of the polymeric chains are terminated with a thiol group or a thiocarbonylthio-containing group —S—(C═S)—Z.

The second article is the reaction product of the first article described above and the ethylenically unsaturated monomers in the presence of actinic radiation (e.g., UV radiation). The second article has a plurality of polymeric groups of Formula (II) covalently bonded to the inorganic oxide coating through a condensation reaction with a silyl group (—Si(R¹)ₙ(R²)₃₋ₙ).

Z—(C═S)—S—(POLY)-Q¹-(L)ₚ-[Si(R¹)ₙ(R²)₃₋ₙ]ₘ (II)

Groups Z, Q¹, L, R¹, and R² as well as the variables p, n, and m are the same as those described above for Formula (I). POLY is the polymeric reaction product of one or more ethylenically unsaturated monomers and is typically water soluble or miscible.

Any suitable ethylenically unsaturated monomers can be used to form POLY. POLY can be a homopolymer, copolymer, terpolymer, block copolymer, and the like. POLY typically has at least one type of reactive functional group. In many embodiments, the reactive functional group is selected to be an azido group, amino group, hydroxy group, carboxylic acid group, carbon-carbon double bond, carbon-carbon triple bond, hydrazone group, tetrazine group, oxirane (epoxy) group, aldehyde group, ketone group, or the like.

In a first embodiment, POLY is formed from a monomer composition that includes a monomer having a reactive functional group. That is, the monomer composition includes a first monomer having a reactive functional group and can further include a second monomer that is water soluble but that does not have a reactive functional group. In this embodiment, the first monomer typically contains an ethylenically unsaturated group and further contains a reactive functional group such as any of those listed above. In an example of the first embodiment, the monomer composition includes a first monomer with an amino group plus an optional second water-soluble monomer. In another example of the first embodiment, the monomer composition includes a first monomer with an azido group plus an optional second water-soluble monomer.

In a second embodiment, the monomer composition can include a first monomer that does not include one of the reactive functional groups listed above but has a bromo group that can be later reacted to form one of the reactive functional group. In this second embodiment, the monomer composition includes a first monomer that has an ethylenically unsaturated group plus a bromo group and can further include a second monomer that is water soluble but that does not have a reactive functional group. The polymerization process can occur either before or after reaction of the bromo group to form the reactive functional group. In one example, a bromo-containing monomer or a bromo-containing polymer can be reacted with sodium azide to form a monomer or a polymer with an azido group.

If the second articles are used for biochemical analysis, POLY is often selected to have azido groups or amino groups. The amino groups in the polymer (POLY) can react, for example, with an ester compound. In some examples, the ester compound can be a fluorescent compound or a bioactive material. The azido groups in the polymer (POLY) can undergo a click chemistry reaction with an unsaturated compound such that the unsaturated compound is covalently linked to the polymer. The unsaturated compound can have, for example, a carbon-carbon triple bond, an unsaturated bicyclic olefinic group, or an acrylamido group. In some examples, the unsaturated compound can be a fluorescent compound or a bioactive material.

If the monomer composition contains an amino group, the amino-containing monomer can have a primary amino group, secondary amino group, tertiary amino group, or quaternary group. Primary amino-containing monomers include, but are not limited to, vinyl amine, allyl amine, aminoalkyl (meth)acylamide (e.g., 2-aminoethyl (meth)acrylamide and 3-aminopropyl (meth)acrylamide), aminoalkyl (meth)acrylate (e.g., 2-aminoethyl (meth)acrylate and 3-aminopropyl (meth)acrylate), 2-N-morpholinoalkyl (meth)acrylate, and hydrochloride salts thereof. Secondary amino-containing monomers include, but are not limited to, various alkylaminoalkylene (meth)acrylates such as, for example, 2-(methylamino)ethyl (meth)acylate and salts thereof. Tertiary amino-containing monomers include, but are not limited to, various N,N-dialkylaminoalkyl (meth)acrylates and N,N-dialkylaminoalkyl (meth)acrylamides such as N,N-dimethyl aminoethyl (meth)acrylate, N,N-dimethylaminoethyl (meth)acrylamide, N,N-dimethylaminopropyl (meth)acrylate, N,N-dimethylaminopropyl (meth)acrylamide, N,N-diethylaminoethyl (meth)acrylate, N,N-diethylaminoethyl (meth)acrylamide, N,N-diethylaminopropyl (meth)acrylate, and N,N-diethylaminopropyl (meth)acrylamide, (tert-butylamino)alkyl (meth)acrylate (e.g., tert-butylaminoethyl (meth)acrylate and tert-butylaminopropyl (meth)acrylate), (tert-butylamino)alkyl (meth)acrylamide (e.g., tert-butylaminoethyl (meth)acrylate and tert-butylaminopropyl (meth)acrylamide), and salts thereof. Quaternary amino-containing monomers include, but are not limited to, methacryloylaminopropyl trimethylammonium chloride, diallyldimethylammonium chloride, and 2-acryloxyalkyltrimethylammonium chloride.

If the monomer composition contains an azido-containing monomer, the azido-containing monomer is often of Formula (III)

CH₂═CR²¹—(C═O)—X¹—[R²²-Q¹⁰]_{y}—R²³ (III)

In Formula (III), the group R²¹ is hydrogen or methyl and the group X¹ is —NH— or —O—. The variable y is equal to 0 or 1, the group R²² is an alkylene or a heteroalkylene having at least one oxygen heteroatom (i.e., ether or polyether group), group Q¹⁰ is —(C═O)—X²— or —NH—(C═O)—X²—, and the group X² is —NH— or —O—. Group R²³ is either (a) an ether or polyether group terminated with an azido group or (b) a branched hetero-hydrocarbon group terminated with two or more azido groups. If R²³ is branched, the hetero-hydrocarbon group has a branching location and comprising (i) an ether or polyether group before the branching location and (ii) an ether or polyether group in each branch after the branching location.

In Formula (III), the variable y is equal to 0 to 1. When the variable y is equal to 1, the monomer of Formula (III) is of Formula (III-1). When the variable y is equal to 0, the monomer of Formula (III) is of Formula (III-2).

CH₂═CR²¹—(C═O)—X¹—R²²-Q¹⁰-R²³ (III-1)

CH₂═CR²¹—(C═O)—X¹—R²³ (III-2)

When R²¹ is hydrogen, the azido-containing monomer has a polymerizable group that is either an acryloyloxy group (CH₂═CH—(C═O)—O—) when X¹ is —O— or an acryloylamido group (CH₂═CH—(C═O)—NH—when X¹ is —NH—. When R²¹ is methyl, the azido-containing monomer has a polymerizable group that is either a methacryloyloxy group (CH₂═C(CH₃)—(C═O)—O—) when X¹ is —O— or methacryloylamido group (CH₂═C(CH₃)—(C═O)—NH—) when X¹ is —NH—.

In Formulas (III) and (III-1), the group R²² is a (hetero)alkylene. If R²² is an alkylene, it often has 1 to 10, 1 to 6, 2 to 6, 2 to 4, 3, or 2 carbon atoms. If R²² is a heteroalkylene group, it can have 1 to 5 oxygen heteroatoms and often contains 2 to 10 carbon atoms. In some embodiments, the alkylene is —CH₂CH₂—, —CH₂CH₂CH₂—, or —C(CH₃)₂— and the heteroalkylene is —(CH₂—CH₂—O)ₓ—CH₂CH₂— or —(CH₂—CH₂—CH₂—O)ₓ—CH₂CH₂—CH₂— where x is 1, 2, or 3.

In Formulas (III) and (III-1), the group Q¹⁰ can be —(C═O)—X²— or —NH—(C═O)—X²— with X² being —O— or —NH—. That is, Q¹⁰ can be —(C═O)—O—, —(C═O)—NH—, or —NH—(C═O)—O—, or —NH—(C═O)—NH—.

Group R²³ in any of the above Formulas (III), (III-1) and (III-2) is either (a) an ether or polyether group terminated with an azido group or (b) a branched hetero-hydrocarbon group terminated with two or more azido groups. The hetero-hydrocarbon group has a branching location and contains (i) an ether or polyether group before the branching location and (ii) an ether or polyether group in each branch after the branching location. Thus, R²³ typically contains at least one azido group. In many embodiments, group R²³ contains 1 or 2 azido groups.

Example R²³ groups that are an ether or polyether group terminated with an azido group are often of formula —R²⁴—(O—R²⁴)_{b}—O—R²⁴—N₃ where each R²⁴ is an alkylene and the variable b is an integer in a range of 0 to 40. In some embodiments, each R²⁴ is either ethylene or propylene. The variable b can be 0, at least 1, at least 2, at least 3, at least 4, at least 5, at least 6, at least 8, or at least 10 and up to 40, up to 36, up to 32, up to 30, up to 28, up to 24, up to 20, up to 16, up to 12, up to 10, up to 8, up to 6, or up to 4.

Other example R²³ groups have two or more azido groups and include a branching group. Some branched R²³ groups are of formula —R²⁵—O—(R²⁵—O)ₖ—R²⁵—N[—R²⁵—O—(R²⁵—O)_{q}R²⁵—N₃]₂ where each R²⁵ is independently an alkylene, k is an integer in a range of 0 to 30 and q is an integer in a range of 0 to 10. The alkylene R²⁵ usually has 1 to 4 carbon atoms and is often either ethylene or propylene. The variable k is equal to at least 0, at least 1, at least 2, at least 4, or at least 10 and up to 30, up to 24, up to 20, up to 18, up to 16, up to 14, up to 12, or up to 10. The variable q is equal to at least 0, at least 1, at least 2, at least 3, or at least 4 and up to 10, up to 8, up to 5, or up to 4.

Other branched R²³ groups are of formula —R²⁶—O—(R²⁶—O)_{w}—R²⁶—(C═O)—NH—CH[—R²⁶—O—R²⁶—(C═O)—NH—R²⁶—O—(R²⁶—O)ᵥ—R²⁶—N₃]₂ where each R²⁶ is independently an alkylene, w is in integer in a range of 0 to 30, and v is an integer in a range of 0 to 10. The alkylene R²⁶ usually has 1 to 4 carbon atoms and is often either ethylene or propylene. The variable w is equal to at least 0, at least 1, at least 2, at least 4, or at least 10 and up to 30, up to 24, up to 20, up to 18, up to 16, up to 14, up to 12, or up to 10. The variable v is equal to at least 0, at least 1, at least 2, at least 3, or at least 4 and up to 10, up to 8, up to 5, or up to 4.

In some embodiments of Formulas (III) and (III-1), X¹ is equal to —O— and Q¹⁰ is equal to —NH—(C═O)—X²— where X² is either —NH— or —O—. The azido-containing monomer is of Formula (III-A).

CH₂═CR²¹—(C═O)—O—R²²—NH—(C═O)—X²—R²³ (III-A)

The azido-containing monomers of Formula (III-A) can be formed, for example, by reaction of an isocyanato-containing monomer with an azido-containing compound having a hydroxy or —NH₂ group that can react with the isocyanato group. The azido-containing compound is typically of formula HX²—R²³ where X² and R²³ is the same as defined above. This reaction is shown in Reaction Scheme C.

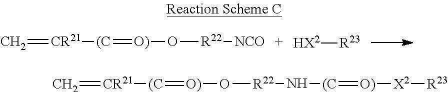

Some example compounds of monomers of formula CH₂═CR²¹—(C═O)—O—R²²—NCO include, but are not limited to, CH₂═CR²¹—(C═O)—O—CH₂CH₂—NCO, CH₂═CR²¹—(C═O)—O—CH₂CH₂CH₂—NCO, and CH₂=CR²¹—(C═O)—O—(CH₂—CH₂—O)_{b}—CH₂CH₂—NCO where the variable b is equal to 1, 2 or 3. The group R²¹ is the same as described above for Formula (III).

Compounds of HX²—R²³ can be of two different types. In the first type, R²³ is an ether or polyether group terminated with an azido group and X² is —O— or —NH—. In the second type, R²³ is a branched hetero-hydrocarbon group terminated with two or more azido groups, the hetero-hydrocarbon group having a branching location and comprising (i) an ether or polyether group before the branching location and (ii) an ether or polyether group in each branch after the branching location.

Examples of the first type of HX²—R²³ compounds are of formula HX²—R²⁴—(O—R²⁴)_{b}—R⁴—N₃ where X² is —O— or —NH—, each R²⁴ is an alkylene and the variable b is an integer in a range of 0 to 40. Some more specific examples include, but are not limited to, H₂N—CH₂CH₂—(O—CH₂CH₂)_{b}—O—CH₂CH₂—N₃ and HO—CH₂CH₂—(O—CH₂CH₂)_{b}—O—CH₂CH₂—N₃ where p ranges from 0 to 40.

Some examples of the second type of HX²—R²³ compounds are of formula HX²—R²⁵—O—(R²⁵—O)ₖ—R²⁵—N[—R²⁵—O(R²⁵O)_{q}R²⁵—N₃]₂ where X² is —O— or —NH—, each R²⁵ is independently an alkylene, k is an integer in a range of 0 to 30, and q is an integer in a range of 0 to 10. The alkylene R²⁵ usually has 1 to 4 carbon atoms and is often either ethylene or propylene. More specific examples include, but are not limited to, compound of formula H₂N—CH₂CH₂—O—(CH₂CH₂—O)ₖ—CH₂CH₂—N[CH₂CH₂—O—(CH₂CH₂—O)_{q}—CH₂CH₂—N₃]₂ where k is an integer in a range of 0 to 30 and q is an integer in a range of 0 to 10.

Other examples of HX²—R²³ compounds of the second type are of formula HX²—R²⁶—O—(R²⁶—O), —R²⁶—(C═O)—NH—CH[—R²⁶—O—R²⁶—(C═O)—NH—R²⁶—O—(R²⁶—O)ᵥ—R²⁶—N₃]₂ where X² is —O— or —NH—, each R²⁶ is independently an alkylene, w is in integer in a range of 0 to 30 and v is an integer in a range of 0 to 10. The alkylene R²⁶ usually has 1 to 4 carbon atoms and is often either ethylene or propylene. More specific examples include, but are not limited to, compound of formula H₂N—CH₂CH₂—O—(CH₂CH₂—O)_{W}—CH₂CH₂—(C═O)—NH—CH[CH₂CH₂—O—CH₂CH₂—(C═O)—NH—CH₂CH₂—O—(CH₂CH₂—O), —CH₂CH₂—N₃]₂ where w is in integer in a range of 0 to 30 and v is an integer in a range of 0 to 10.

In other embodiments of Formulas (III) and (III-1), X¹ is —NH—, Q¹⁰ is —(CO)—X²—, and X² is —O— or —NH—. The azido-containing monomer is of Formula (III-B).

CH₂=CR²¹—(C═O)—NH—R²²—(C═O)—X²—R²³ (III-B) In many embodiments, R²¹ is hydrogen and R²² is an alkylene such as —C(CH₃)₂—. That is, the azido-containing monomer of Formula (III-B) is often CH₂═CH—(C═O)—NH—C(CH₃)₂—(C═O)—X²—R²³.

The azido-containing monomers of Formula (III-B) such as those of formula CH₂═CH—(C═O)—NH—C(CH₃)₂—(C═O)—X²—R²³ can be formed by reaction of vinyl azlactone with an azido-containing compound having a hydroxy or —NH₂ group that can react with the azlactone group as shown in Reaction Scheme D. The azido-containing compound is typically of formula HX²—R²³ as described above for use in Reaction Scheme C.

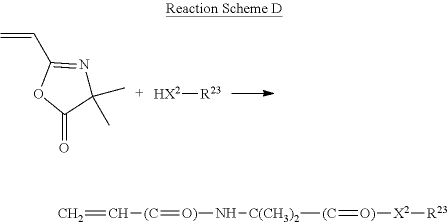

Although the azido-containing monomer is usually of Formula (III-1), it can be of Formula (III-2) as described above.

CH₂=CR²¹—(C═O)—X¹—R²³ (III-2)

Monomers of Formula (III-2) can be formed, for example, by reaction of (meth)acrylate anhydride with an azido-containing compound of formula HX²—R²³ as described above. That is, both X¹ and X² are either hydrogen or methyl. This reaction is shown in Reaction Scheme D. The group X¹ in Formula (III-2) is the same as group X² in the compound HX²—R²³.

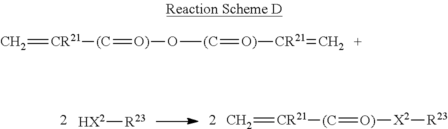

Still other azido-containing monomers are of Formula (IV).

CH₂=CR³²—(C═O)—NH—R³⁰—N₃ (IV)

In Formulas (IV), group R¹ is hydrogen or methyl and group R³⁰ is an alkylene or a group of formula —R³¹—NH—(C═O)—R³¹—N₃ where each R³¹ is independently an alkylene have 1 to 10 carbon atoms. In some examples, the azido-containing monomers of Formula (IV) are CH₂═CH—(C═O)—NH—CH₂CH₂CH₂—N₃ or CH₂═CH—(C═O)—NH—CH₂CH₂CH₂CH₂CH₂—NH—(C═O)—CH₂—N₃. Unlike the azido-containing monomers of Formula (III), these monomers do not contain ether or polyether groups.

The monomers of Formula (IV) can be formed from the corresponding bromo-containing monomer CH₂=CR³²—(C═O)—NH—R³⁰—Br by reacting with sodium azide. Alternatively, this conversion reaction can occur after polymerization. Such monomers and polymers are further described, for example, in U.S. Patent Applications 2020/0131285 (Brown et al.) and 2019/0233890 (George et al.) as well as in U.S. patent Ser. No. 10/577,439 (Brown et al.).

The polymeric group POLY in Formula (II) above contains monomeric units of Formula (III-M) or (IV-M).

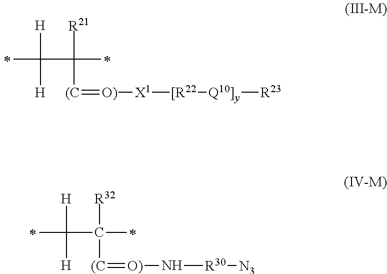

These monomeric units can be formed directly from monomers of Formula (III) or (IV).
Alternatively, these monomeric units can be formed by conversion of other precursor monomers or precursor monomeric units. For example, the monomeric units can be formed by reaction of a precursor bromo-containing monomer or a precursor bromo-containing monomeric unit with sodium azide. The polymeric group POLY can be a homopolymer, copolymer, terpolymer, block copolymer, or the like.

The azido-containing monomer or a bromo-containing monomer precursor can be polymerized with a second monomer having an ethylenically unsaturated group to form the polymer (POLY) in Formula (II). Any known second monomer having an ethylenically unsaturated group can be used. For example, the second monomer can be a (meth)acrylate monomer, a (meth)acrylamide monomer, a vinyl monomer, a styryl monomer or an allyl monomer. In many embodiments, the second monomer is a (meth)acrylate or (meth)acrylamide monomer.

In some embodiments, the azido-containing monomer is polymerized with a second monomer that can result in the formation of a polymer (POLY) that is water soluble or water swellable. For example, to enhance water solubility, the second monomer can have an anionic group, a cationic group, a hydrogen bond acceptor group, a hydrogen bond donor group, or a combination thereof. Other monomers that do not have any of these groups can also be used, if desired.

Although other monomers can be used, the second monomers are often selected to be (meth)acrylamides. Examples include, but are not limited to, (meth)acrylamide, N,N-dimethyl(meth)acrylamide, N-ethylacrylamide, N-hydroxymethylacrylamide, N-hydroxyethylacrylamide, and 2-Acrylamido-2-methylpropane sulfonic acid (AMPS).

Other examples of preferred second monomers include, but are not limited to, N-vinyl-2-pyrrolidinone, 2-hydoxyethyl (meth)acrylate, 2-hydroxypropyl (meth)acrylate, methacryloylaminopropyl trimethylammonium chloride, and polyethylene glycol mono(meth)acrylates (such as methoxypolyethylene glycol (350) monomethacrylate, available under trade designation CD 550 from Sartomer (Exton, PA, USA)).

The amount of the azido-containing monomer of Formula (III) or (IV) used to form the polymer can be any desired amount ranging from 0.1 to 100 mole percent based on total moles of monomeric units in the polymer. The amount can be at least 0.1 mole percent at least 0.5 mole percent, at least 1 mole percent, at least 2 mole percent, at least 5 mole percent, at least 10 mole percent, at least 15 mole percent, at least 20 mole percent, at least 25 mole percent, at least 30 mole percent, at least 35 mole percent, or at least 40 mole percent and up to 100 mole percent, up to 90 mole percent, up to 80 mole percent, up to 70 mole percent, up to 60 mole percent, or up to 50 mole percent. The azido-containing monomer is often of Formula (III). The monomeric units that do not contain an azido group can be derived from one or more of the second monomers described above or from any other known monomer.

In some embodiments, POLY contains 0.1 to 50 mole percent monomeric units of Formula (III-M) or Formula (IV-M) and 50 to 99.9 mole percent monomeric units derived from any second monomer such as those described above. For example, the polymer can contain 0.5 to 50 mole percent, 1 to 50 mole percent, 0.1 to 40 mole percent, 0.5 to 40 mole percent, 1 to 40 mole percent, 2 to 40 mole percent, 5 to 40 mole percent, 0.1 to 30 mole percent, 0.5 to 30 mole percent, 1 to 30 mole percent, 2 to 30 mole percent, 5 to 30 mole percent, 0.1 to 20 mole percent, 0.5 to 20 mole percent, 1 to 20 mole percent, 2 to 20 mole percent, 5 to 20 mole percent, 0.1 to 10 mole percent, 0.5 to 10 mole percent, 1 to 10 mole percent, 0.1 to 5 mole percent, 0.5 to 5 mole percent, or 1 to 5 mole percent of the azido-containing monomeric units of Formula (III-M) or Formula (IV-M) with the remainder derived from one or more of the second monomers described above. In these embodiments, the azido-containing monomeric units are often of Formula (III-M).

The reaction to form the polymeric material (POLY) typically includes contacting the portion of the first article in contact with the monomers. The reaction mixture often contains the monomers and a non-reactive liquid. The non-reactive liquid is often water, a polar solvent, or a mixture thereof. Suitable polar solvents include, for example, dimethylformamide, dimethylsulfoxide, N-methylpyrrolidinone, dimethylacetamide, tetramethylurea, N,N′-dimethylpropylene urea, methanol, and the like. The polymerization reaction occurs upon exposure to actinic radiation, particularly to ultraviolet radiation.

The reaction mixture can optionally further include a photoiniferter, other types of photoinitiators, or a thermal initiator that is not bonded to the surface of the inorganic oxide coating. These additional photoiniferters or initiators can promote growth of the brush polymers attached to the inorganic oxide layer but may also result in the formation of polymeric material in the solution that are not attached to the inorganic oxide coating. The amount of the optional photoiniferter or initiator can be controlled to minimize the amount of polymeric material that is formed but not attached to the inorganic oxide coating. Preferably, the photoiniferter in solution has a reactivity comparable to the photoiniferter attached to the inorganic oxide coating. This is achieved by selecting a photoiniferter in solution that has a similar Z group and a similar radical leaving group to the photoiniferter of Formula (I).

Suitable optional photoiniferters are those that do not have a silyl group. Examples of photoiniferters without a silyl group include, but are not limited to, the following compounds:

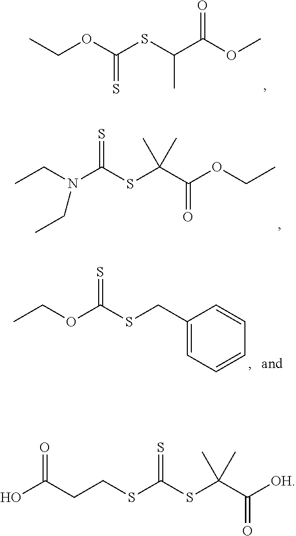

Suitable photoinitiators include, for example, benzoin ethers (e.g., benzoin methyl ether or benzoin isopropyl ether) or substituted benzoin ethers (e.g., anisoin methyl ether). Other exemplary photoinitiators are substituted acetophenones such as 2,2-diethoxyacetophenone or 2,2-dimethoxy-2-phenylacetophenone (commercially available under the trade designation OMNIRAD BDK from IGM Resins (Charlotte, NC, USA)). Still other exemplary photoinitiators are substituted alpha-ketols such as 2-methyl-2-hydroxypropiophenone, aromatic sulfonyl chlorides such as 2-naphthalenesulfonyl chloride, and photoactive oximes such as 1-phenyl-1,2-propanedione-2-(O-ethoxycarbonyl)oxime. Other suitable photoinitiators include, for example, 1-hydroxycyclohexyl phenyl ketone (commercially available under the trade designation OMNIRAD 184), bis(2,4,6-trimethylbenzoyl)phenyl phosphine oxide (commercially available under the trade designation OMNIRAD 819), 1-[4-(2-hydroxyethoxy)phenyl]-2-hydroxy-2-methyl-1-propane-1-one (commercially available under the trade designation Omnirad 2959), 2-benzyl-2-dimethylamino-1-(4-morpholinophenyl)butanone (commercially available under the trade designation OMNIRAD 369), 2-methyl-1-[4-(methylthio)phenyl]-2-morpholinopropan-1-one (commercially available under the trade designation OMNIRAD 907), and 2-hydroxy-2-methyl-1-phenyl propan-1-one (commercially available under the trade designation DAROCUROMNIRAD 1173. from iGM Resins, (Charlotte, NC, USA).

Suitable thermal initiators include, for example, various azo compound such as those commercially available under the trade designation VAZO from Chemours Co. (Wilmington, DE, USA) including VAZO 67, which is 2,2′-azobis(2-methylbutane nitrile), VAZO 64, which is 2,2′-azobis(isobutyronitrile), VAZO 52, which is (2,2′-azobis(2,4-dimethylpentanenitrile), and VAZO 88, which is 1,1′-azobis(cyclohexanecarbonitrile), VAZO 68 WSP, which is 4,4′-azobis-(4-cyanopentanoic acid), and VAZO 56 WSP, which is 2,2′-azobis(2-methylpropionamidine) dihydrochloride; various peroxides such as benzoyl peroxide, cyclohexane peroxide, lauroyl peroxide, di-tert-amyl peroxide, tert-butyl peroxy benzoate, di-cumyl peroxide, and peroxides commercially available from Atofina Chemical, Inc. (Philadelphia, PA, USA) under the trade designation LUPERSOL (e.g., LUPERSOL 101, which is 2,5-bis(tert-butylperoxy)-2,5-dimethylhexane, and LUPERSOL 130, which is 2,5-dimethyl-2,5-di-(tert-butylperoxy)-3-hexyne); various hydroperoxides such as tert-amyl hydroperoxide and tert-butyl hydroperoxide; and mixtures thereof.

The optional photoiniferter or initiator is often added in an amount in a range of 0 to 5 weight percent based on the total weight of the monomers in the reaction mixture. The amount is often at least 0.1, at least 0.5, at least 1, at least 1.5, at least 2, at least 2.5 and up to 5, up to 4.5, up to 4, up to 3.5, up to 3, up to 2.5, or up to 2 weight percent based on the total weight of monomers in the reaction mixture.

In some embodiments, the attached polymeric material of Formula (II) is further reacted to replace the terminal group —S—(C═S)—Z with an alternative group. This replacement can be accomplished, for example, to replace this group with a more inert group or with a group that does not contribute as much color to the polymeric material. That is, the attached polymeric material can be of Formula (V).

R⁴⁰—(POLY)-Q¹-(L)ₚ-[Si(R¹)ₙ(R²)₃₋ₙ]ₘ (V)

In Formula (V), the groups Q¹, L, R¹, and R² as well as the variable n, m, and p are the same as described above in Formula (II). Group R⁴⁰ is hydrogen, —SH, —S—CH₂R⁴¹—(C═O)—OR⁴², —C(R⁴³)R⁴⁴—C≡N, or —C(CH₃)R⁴⁵—C≡N. In the group —S—CH₂CHR⁴¹—(C═O)—OR⁴², R⁴¹ is hydrogen or methyl and R⁴² is an alkyl such as an alkyl having 1 to 10, 1 to 6, 1 to 4, or 1 to 3 carbon atoms. In the group —C(R⁴³)R⁴⁴—C≡N, R⁴³ and R⁴⁴ are each an alkyl (e.g., an alkyl having 1 to 3 carbon atoms) or combine together to form a cycloalkyl group (e.g., a cycloalkyl having 5 or 6 carbon atoms). In some embodiments, R⁴³ is methyl and R⁴⁴ is methyl or ethyl. In other embodiments R⁴³ and R⁴⁴ combine to form a cyclohexyl group. In the group —C(CH₃)R⁴⁵—C≡N, R⁴⁵ is an acidic group such as —CH₂CH₂—(C═O)—OH or a basic group such as —C(NH)(NH₃⁺)X⁻ where X⁻ is a halide such as chloride.

The replacement of the —S—(C═S)—Z group with hydrogen can be accomplished as described, for example, by Carmean et al. in ACS Macro Lett., 2017, 6, 185-19. These authors describe a method in which long wavelength ultraviolet radiation (e.g., 365 nm) is used in the presence of a hydrogen donor such as N-ethylpiperidine hypophosphate.

The replacement of the —S—(C═S)—Z groups with a —SH group can be accomplished by treatment of the polymer brush with a nucleophilic base, such as a primary amine. The resulting —SH group can subsequently be reacted with an alkyl (meth)acrylate to introduce a group —S—CH₂CR⁴¹—(C═O)—OR⁴².

The replacement of the —S—(C═S)—Z group with a groups such as —C(R⁴³)R⁴⁴—C≡N and —C(CH₃)R⁴⁵—C≡N can occur if thermal radical initiators such as those commercially available under the trade designation VAZO are added in excess to the reaction mixture used to form the polymeric brushes (POLY) after polymerization has completed.

FIG. 1B is representative of one embodiment of the second article. The second article differs from the first article of FIG. 1A by replacement of the attached photoiniferter groups of Formula (I) with attached polymeric material of Formula (II). The polymeric material of Formula (II), which corresponds to 50 in FIG. 1B, is attached to the inorganic oxide layer surface 22 in the wells 32.

FIG. 2B is representative of a second embodiment of the second article. The second article differs from the first article of FIG. 2A by replacement of the attached photoiniferter groups of Formula (I) with attached polymeric material of Formula (II). The polymeric material of Formula (II), which corresponds to 600 in FIG. 2B, is attached to the surface 210 of the inorganic oxide coating 200 on the structures 300.

In some embodiments, the second article further includes an optional adhesive layer opposite the surface with the structures. The adhesive layer can be used to attach the second article to another substrate such as a glass, ceramic, and another polymeric layer. This can be done, for example, to decrease the flexibility of the second article. The adhesive layer is typically selected to have a low auto-fluorescence is the second article is used for chemical or biochemical analysis. The adhesive can be, for example, an optically clear adhesive such as those commercially available from 3M Company (e.g, 3M Optically Clear Adhesive 8171). Other suitable adhesives include polyisobutylene based adhesives. The adhesive layer can be positioned next to another substrate or can be positioned adjacent to a release liner for later placement adjacent to the other substrate.

The first articles advantageously include a photoiniferter attached to an inorganic surface of a substrate having an inorganic oxide layer on a polymeric film. The substrates can be patterned and can be formed using roll-to-roll processing. These first articles are particularly advantageous for the formation of the second articles with polymeric materials attached to the substrate. For example, the second articles have attached brush polymers that can have a higher local concentration of reactive groups in the wells or on the posts compared to polymers that lies solely at the bottom of the well or the top of the posts. That is, the brush polymers extend away from the bottom surface of the well or the top surface of the posts and can have any desired concentration of the reactive groups. Further, the use of photoiniferters attached to the wells or posts allow for greater control over the length and uniformity of the polymeric brushes that are formed and allow for the formation of block copolymers, if desired. Further, the photoiniferter chemistry tends to be tolerant of a variety of different types of reactive function groups.

### Method of Using the Second Articles

The second articles described above that include brush polymers with reactive functional groups can be reacted with other compounds. For example, in many second articles, the brush polymer has an azido group. That is, the brush polymers are prepared using an azido-containing monomer such as those in Formula (III) or (IV) above. The azido groups can react with an unsaturated compound capable of undergoing a click chemistry reaction with an azido group.

Such compounds often have a terminal —C≡CH group, a carbon-carbon triple bond (—C≡C—) such as in a cyclic group having eight ring members, a carbon-carbon double bond (—C═C—) as in an unsaturated bicyclic olefinic group having seven ring members, or an acrylamido group. The click chemistry reaction results in the covalent attachment of the unsaturated compound to the polymer.

There are numerous commercially available compounds having a group that can undergo a click chemistry reaction with an azido group. Additionally, there are several companies that specialize in adding a group capable of undergoing a click chemistry reaction to a specifically selected compound such a polynucleotide (e.g., oligonucleotide) or a polypeptide (e.g., oligopeptide), carbohydrates (e.g. monosaccharides, oligosaccharides or polysaccharides), or other bioactive molecules (e.g. biotin). Such companies include, for example, JPT Peptide Technology (Berlin, Germany) Integrated DNA Technologies (Coralville, IA, USA), ThermoFisher Scientific (Waltham, MA, USA), Jena Biosciences (Jena, Germany), CarboSynth Ltd (Berkshire, United Kingdom), and BroadPharm (San Diego, CA, USA).

Further, there are several companies that provide fluorescent dyes having a carbon-carbon triple bond. These include fluorescent dyes commercially available under the trade designation AFDye (e.g., AFDye 350 alkyne and AFDye 488 DBCO), Cy5 alkyne, Oregon Green 488 alkyne, and TAMRA alkyne from Click Chemistry Tools (Scottsdale, AZ, USA). Similar fluorescent dyes are commercially available from ThermoFisher Scientific (Waltham, MA, USA), BroadPharm (San Diego, CA, USA), and Conju-Probe, LLC (San Diego, CA, USA).

The second article can be part of a device for chemical and/or biochemical analysis. For example, the second article can be used in a device for DNA sequencing.

### EXAMPLES

Unless otherwise noted, all parts, percentages, ratios, etc. in the Examples and the rest of the specification are by weight. Unless otherwise indicated, all other reagents were obtained, or are available from fine chemical vendors such as VWR, Radnor, PA, or may be synthesized by known methods. Table 1 (below) lists materials used in the examples and their sources.

TABLE 1
Materials List

| DESIGNATION | DESCRIPTION | SOURCE |
|---|---|---|
| 3-mercaptopropyl trimethoxysilane | 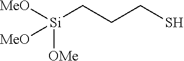 [SMILES: CO[Si](CCCS)(OC)OC] | Gelest Morrisville, PA, U.S.A. |
| Allyl 2- bromopropionate | 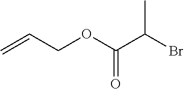 | ABCR Karlsruhe, Germany |
| VAZO-67 | 2,2′-Azobis(2-methylpropionitrile) | Chemours |
| | | Wilmington, DE, U.S.A. |
| Potassium ethyl xanthate | 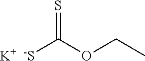 | Alfa Aesar Ward Hill, MA, U.S.A. |
| (3- trimethoxysilyl)propyl 2-bromo-2- methylpropionate | 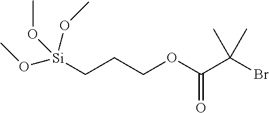 [SMILES: CO[Si](CCCOC(=O)C(C)(C)Br)(OC)OC] | Gelest Morrisville, PA, U.S.A. |
| Sodium diethyldithiocarbamate trihydrate | 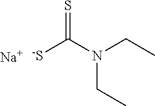 | Alfa Aesar Ward Hill, MA, U.S.A. |
| (p-chloromethyl)phenyl trimethoxysilane | 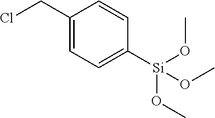 | Gelest, Morrisville, PA, U.S.A. |
| Methyl 2- bromopropionate | 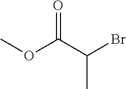 | MilliporeSigma, Burlington, MA, U.S.A. |
| Acrylamide | CH₂═CH—(CO)—NH₂ | Alfa Aesar Ward Hill, MA, U.S.A. |
| 4,4-Dimethyl-2-vinyl- 4H-oxazol-5-one | 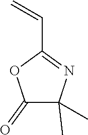 | Isochem North America, Syosset, NY, U.S.A. |
| 11-azido-3,6,9- trioxaundecan-1-amine | 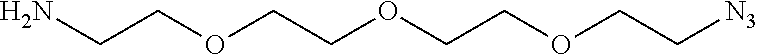 [SMILES: [N-]=[N+]=NCCOCCOCCOCCN] | TCI America Portland, OR, U.S.A. |
| Potassium phosphate | KH₂PO₄ | VWR International LLC, |
| monobasic | | Radnor, PA, U.S.A. |
| Potassium phosphate | K₂HPO₄•3H₂O | MilliporeSigma |
| dibasic trihydrate | | Burlington, MA, U.S.A. |
| PHOTOMER 6210 | Aliphatic urethane diacrylate resin | IGM Resins |
| | | Charlotte, NC, U.S.A. |
| SR238 | 1,6-hexanediol diacrylate | Sartomer (Arkema Group), |
| | | West Chester, PA, U.S.A. |
| SR351 | Trimethylolpropane triacrylate | Sartomer |
| | | West Chester, PA, U.S.A. |
| TPO | 2,4,6-trimethylbenzoyldiphenyl | IGM Resins |
| | phosphine oxide | Charlotte, NC, U.S.A. |
| PVB-30H | Polyvinyl butyral | Kuraray America |
| | | Houston, TX, U.S.A. |
| MELINEX 454 | Polyester film | Tekra |
| | | New Berlin, WI, U.S.A. |
| Acrylamide silane | 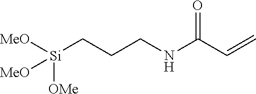 | Prepared as described in Wang et. al., Macromolecules, 2011, 44, 878-885. |
| PE1-A | 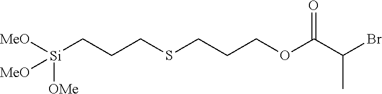 [SMILES: CO[Si](CCCSCCCOC(=O)C(C)Br)(OC)OC] | Prepared as described in Preparative Example 1. |
| PE1-B | 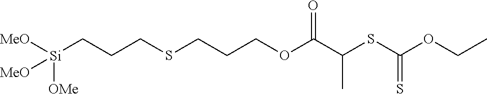 [SMILES: CCOC(=S)SC(C)C(=O)OCCCSCCC[Si](OC)(OC)OC] | Prepared as described in Preparative Example 1. |
| PE2 | 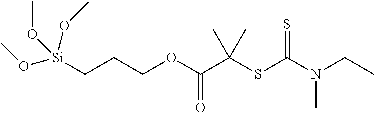 [SMILES: CCN(C)C(=S)SC(C)(C)C(=O)OCCC[Si](OC)(OC)OC] | Prepared as described in Preparative Example 2. |
| PE3 | 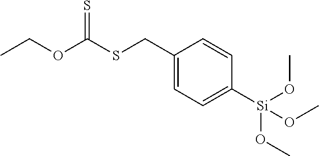 [SMILES: CCOC(=S)SCc1ccc([Si](OC)(OC)OC)cc1] | Prepared as described in Preparative Example 3. |
| PE4 | 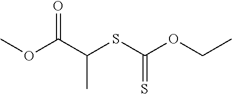 | Prepared as described in Preparative Example 4. |
| PE5 | 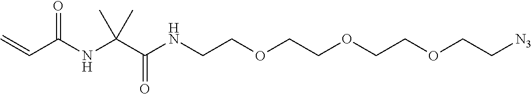 [SMILES: C=CC(=O)NC(C)(C)C(=O)NCCOCCOCCOCCN=[N+]=[N-]] | Prepared as described in Preparative Example 5. |
| Nano-structured film | Poly(ethyleneterephthalate) (PET) film | Prepared as described |
| | with nano-posts capped with silicon carbon | in Preparative Example |
| | oxide. The interstitial spaces between the | 9 (output of all five |
| | posts are coated with release treatment. | steps.) |
| HMDSO | Hexamethyldisiloxane | Gelest |
| | | Morrisville, PA, U.S.A. |
| N-(3-aminopropyl) methacrylamide hydrochloride | 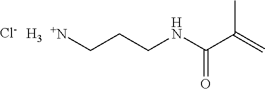 | Polysciences Warrington, PA, U.S.A. |
| AF488 NHS Ester | Succinimidyl ester of fluorescent dye | Thermo Fisher |
| | obtained as ALEXA FLUOR 488 NHS | Scientific |
| | ESTER | Waltham, MA, U.S.A. |
| TE Buffer pH 8 | Buffer solution obtained as | Thermo Fisher Scientific |
| | INVITROGEN TE BUFFER pH 8 | Vilnius, Lithuania |
| Alkyne oligonucleotide | An oligonucleotide labeled with | Integrated DNA |
| having a fluorescein | Fluorescein (Sequence: 5′-Hexynyl- | Technologies (IDT) |
| group | GCG CTG TTC ATT CGC G- | Coralville, IA, U.S.A. |
| | Fluorescein-3′) | |
| PMDETA | N,N,N′,N′,N″- | MilliporeSigma |
| | pentamethyldiethylenetriamine | Burlington, MA, U.S.A. |
| Copper sulfate | (CuSO₄•5H₂O) | Alfa Aesar |
| pentahydrate | | Ward Hill, MA, U.S.A. |
| Sodium ascorbate | Sodium ascorbate | Alfa Aesar |
| | | Ward Hill, MA, U.S.A. |
| DNA LOBIND TUBES | Centrifuge tubes | Eppendorf |
| | | Enfield, CT, U.S.A. |

### Test Methods

### Confocal Microscopy

The nanopatterned samples with fluorescent dyes or oligonucleotides were imaged using a confocal microscope (Zeiss AXIOPLAN 2 with LSM 510 Laser Module, Zeiss, Thornwood, NJ, USA) equipped with an ACHROPLAN 63×/1.4 Oil DIC M27 (FWD=0.19 mm) objective. The film samples were adhered on a 2.5 cm by 7.6 cm (1 inch by 3 inch) microscope slide using a droplet of RESOLVE Microscope Immersion Oil (Cornwell Corp., Riverdale, NJ, USA) and covered with a glass cover slip, onto which another droplet of microscope oil was added. The fluorescent images were then taken using 488 nm laser excitation at 70-75% power and a 505 nm long pass filter. The scanning parameters were set to define a field of view of 40.93 microns×40.93 microns.

### PREPARATIVE EXAMPLES

### Preparative Example 1

### Step 1: Synthesis of PE1-A

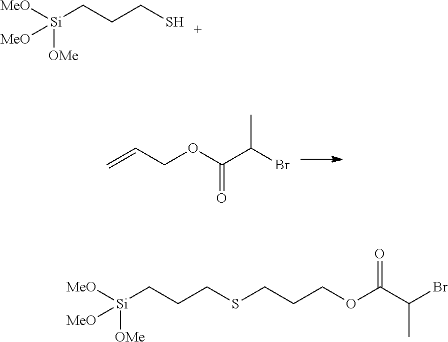

A 100 milliliter (mL) round-bottom flask was charged with 3-mercaptopropyl trimethoxysilane (5.00 grams (g), 25.5 millimoles (mmol)), allyl 2-bromopropionate (5.06 g, 26.2 mmol), VAZO-67 (0.245 g, 1.27 mmol), ethyl acetate (45 g), and methanol (5 g). The flask was equipped with a stir bar, condenser, and capped with a rubber septum. Nitrogen gas was bubbled through the solution via a needle inserted into the septum for 15 minutes. The flask was then immersed in an oil bath held at 80° C. for 15 hours. The solvent was removed by distillation at reduced pressure using a rotary evaporator. Residual starting material was removed by distillation at low pressure (<0.1 torr) and a temperature of 65° C. Isolated 9.67 g (98% yield) of product PE1-A as a clear oil.

### Step 2: Synthesis of PE1-B from PE1-A

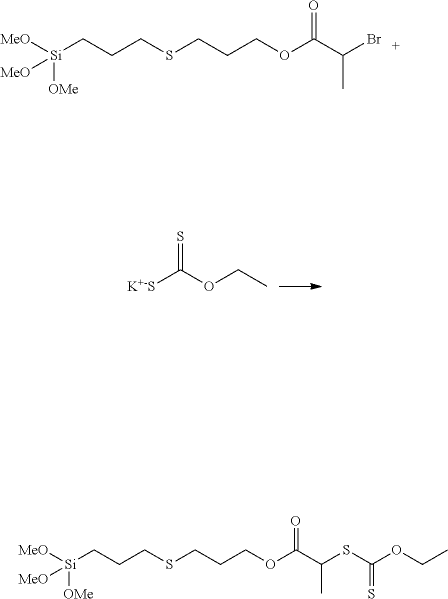

Potassium ethyl xanthate was dried in a vacuum oven held at 60° C. for 1 hour prior to use. Acetone was dried by storing over flame-activated 3 Angstrom molecular sieves for 1 hour immediately prior to use. Under a blanket of nitrogen, a 100 mL round-bottom flask was charged with PE1-A (7.86 g, 20.2 mmol), potassium ethyl xanthate (3.56 g, 22.2 mmol), and acetone (50 g). The suspension was stirred under nitrogen at room temperature for 3 days. The suspension was filtered through a fine glass frit. The resulting solution was further filtered through a 1 micron glass fiber syringe tip filter (Pall Corporation, Port Washington, NY). The solution was then concentrated by distillation at reduced pressure using a rotary evaporator, yielding 8.30 g (95% yield) of product PE1-B as a light yellow oil.

### Preparative Example 2

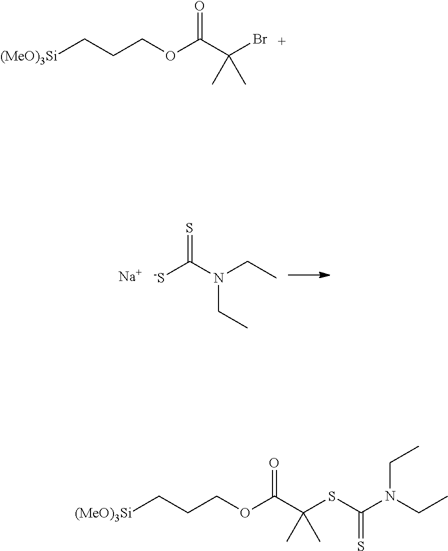

A 25 mL round-bottom flask was charged with sodium diethyldithiocarbamate trihydrate (648 mg, 2.88 mmol), acetone (5.0 g), and flame-activated 3 Angstrom molecular sieves (1.0 g). The flask was septum-capped, and the suspension was stirred under a blanket of nitrogen for 5 hours. Then (3-trimethoxysilyl)propyl 2-bromo-2-methylpropionate (758 mg, 2.30 mmol) was added by syringe. The suspension was stirred at room temperature for 4 days then filtered through a 1 micron glass fiber syringe tip filter. The acetone was evaporated with a stream of nitrogen, and the product was dissolved in dichloromethane (10 g). This solution was filtered through a 0.45 micron PTFE syringe-tip filter (Fisher Scientific, Hampton, NH, USA). The solvent was evaporated with a stream of nitrogen, and the resulting oil was dried further under vacuum, yielding 0.888 g (78% yield) of PE2 as a light yellow oil.

### Preparative Example 3

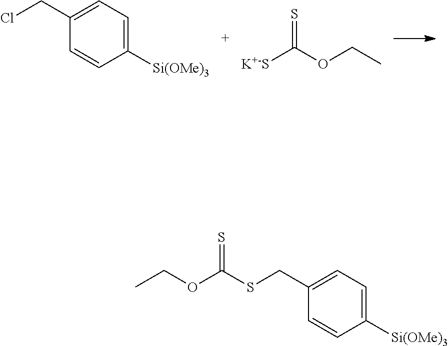

Potassium ethyl xanthate was dried in a vacuum oven held at 60° C. for 1 hour prior to use. Acetone was dried by storing over flame-activated 3 Ångstrom molecular sieves for 1 hour immediately prior to use. A 4 ounce glass jar was charged with (p-chloromethyl)phenyltrimethoxysilane (2.10 g, 8.51 mmol), potassium ethyl xanthate (1.50 g, 9.36 mmol), and acetone (20 mL). The jar was sealed and the suspension was stirred at room temperature overnight. The suspension was then filtered through a 5 micron PTFE syringe tip filter (Whatman, Maidstone, UK) resulting in a clear solution. The acetone was evaporated with a stream of nitrogen, and the resulting oil was dried further under vacuum, yielding 2.57 g (91% yield) of PE3 as a light yellow oil.

### Preparative Example 4

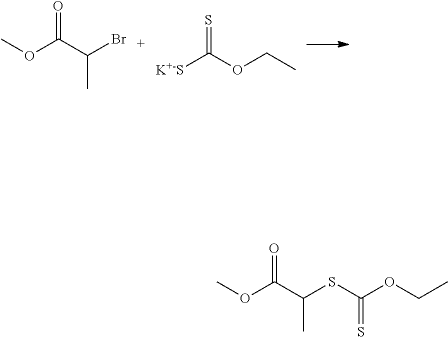

A 100-mL round-bottom flask was charged with methyl 2-bromopropionate (2.00 g, 12.0 mmol), potassium ethyl xanthate (2.02 g, 12.6 mmol), and acetone (10 g). The resulting suspension was stirred for 15 hours at room temperature. The suspension was filtered through a fine glass frit. The resulting solution was further filtered through a 1 micron glass fiber syringe tip filter. The solution was then concentrated by distillation at reduced pressure using a rotary evaporator, yielding 2.39 g (96% yield) of product PE4 as a light-yellow oil.

### Preparative Example 5

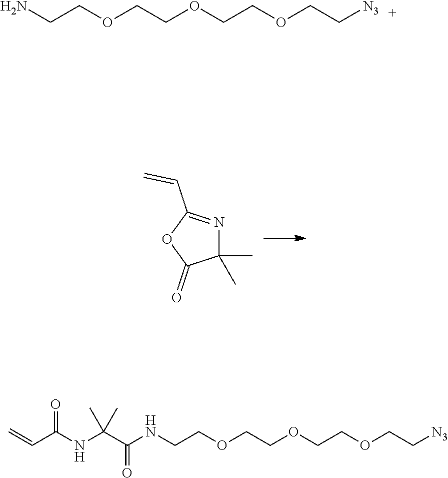

4,4-Dimethyl-2-vinyl-4H-oxazol-5-one (1.0 mL) was added to a flask containing a solution of 11-azido-3,6,9-trioxaundecan-1-amine (1.0 mL) in diethyl ether (5.0 mL). After one hour, the upper phase was removed and diethyl ether (10 mL) was added to the flask. The resulting mixture was stirred overnight and then concentrated under reduced pressure overnight to provide PE5 as a clear oil.

### Preparative Example 6

### 10 mM Potassium Phosphate Buffer of pH 7.0

A 0.1 M potassium phosphate buffer was first prepared by combining 38.5 g of 1 M KH₂PO₄ and 61.5 g of 1 M potassium phosphate dibasic trihydrate. 10 mM phosphate buffer with pH 7.0 was then prepared by mixing 10 g of the 0.1 M potassium phosphate buffer with 90 g of deionized water.

### Preparative Example 7

A resin was prepared by combining and mixing PHOTOMER 6210, SR238, SR351, and TPO in weight ratios of 60/20/20/0.5. After all components were added, the mixture was blended by warming to approximately 50° C. and mixing for 12 hours on a roller mixer until the mixture appeared homogeneous.

### Preparative Example 8

A resin was prepared by combining and mixing PHOTOMER 6210, SR238 and TPO in weight ratios of 75/25/0.5. After all components were added, the mixture was blended by warming to approximately 50° C. and mixing for 12 hours on a roller mixer until the mixture appeared homogeneous.

### Preparative Example 9

### Step 1: Preparation of Unmodified Nano-Structured Film

A nano-structured film was prepared by die coating the resin of Preparative Example 7 onto a conventional 75 μm thick biaxially oriented polyethylene terephthalate (PET) film. The PET film is referred to as the polymeric support film or support film. The side of the PET film that contacted the resin was primed with a thermoset acrylic polymer (RHOPLEX 3208 obtained from Dow Chemical, Midland, MI). The primed film was then coated with the liquid resin of Preparative Example 7, which is a mixture of various (meth)acrylate monomers having multiple (meth)acryloyl groups. The liquid resin layer of the coated film was pressed against a nanostructured nickel surface attached to a steel roller controlled at 60° C. using a rubber-covered roller at a speed of 15.2 meters/min. The nanostructured nickel surface consists of a single 15 cm by 15 cm patterned area with regularly spaced well features of 1500 nm diameter and 350 nm depth. The coating thickness of the resin of Preparative Example 7 on the support film was sufficient to fully wet the nickel surface and form a rolling bead of resin as the coated film was pressed against the nano-structured nickel surface. The coated film was exposed to radiation through the support film from two Fusion UV lamp systems (obtained under the trade designation “F600” from Fusion UV Systems, Gaithersburg, MD) fitted with D bulbs both operating at 142 W/cm² while in contact with the nanostructured nickel surface. After peeling the coated film from the nanostructured nickel surface, the nanostructured side of the coated film was exposed again to radiation from the Fusion UV lamp system fitted with a D bulb operating at 142 W/cm².

### Step 2: Release Treatment

A release layer with a methylated surface was formed according to methods described in U.S. Pat. No. 6,696,157 (David et al.) and U.S. Pat. No. 8,664,323 (Iyer et al.) and U.S. Patent Publication No. 2013/0229378 (Iyer et al.). It was applied to the nano-structured film in a parallel plate capacitively coupled plasma reactor to create a nano-structured release film. The chamber has a central cylindrical powered electrode with a surface area of 1.7 m² (18.3 ft²). After placing the nano-structured film and the underlying polymeric support film (i.e., the product of step 1) on the powered electrode with the nano-structured film exposed, the reactor chamber was pumped down to a base pressure of less than 1.3 Pa (2 mTorr). O₂ gas was flowed into the chamber at a rate of 1000 standard cubic centimeters per minute (SCCM). Treatment was carried out using a plasma enhanced chemical vapor deposition (CVD) method by coupling radio frequency (RF) power into the reactor at a frequency of 13.56 megahertz (MHz) and an applied power of 2000 watts. Treatment time was controlled by moving the nano-structured film on the support film through the reaction zone at rate of 9.1 meters/min (30 ft/min) resulting in an approximate exposure time of 10 seconds. After completing the deposition, RF power was turned off and gases were evacuated from the reactor. Following the 1st treatment, a 2nd plasma treatment was carried out in the same reactor without returning the chamber to atmospheric pressure. HMDSO gas was flowed into the chamber at approximately 1750 SCCM to achieve a pressure of 9 mTorr. 13.56 MHz RF power was subsequently coupled into the reactor with an applied power of 1000 W. The nano-structured film on the support film was then carried through the reaction zone at a rate of 9.1 meter/min (30 ft/min) resulting in an approximate exposure time of 10 seconds. At the end of this treatment time, the RF power and the gas supply were stopped, and the chamber was returned to atmospheric pressure.

### Step 3: Planarizing Layer

A solution of 4% by weight PVB-30H solution in isopropanol was die coated in a roll-to-roll process onto the nano-structured release film of step 3 with a slot die at a rate of 0.025 m/s. The solution was coated 15.3 cm wide and pumped with a Harvard (Harvard Apparatus, Holliston, MA, USA) syringe pump at a rate of 2.97 SCCM. The coating was dried at 65° C. for 4 minutes to create a planarized film.

### Step 4: Etch

Reactive ion etching was carried out on the planarized film in the same home-built reactor chamber used to deposit the plasma enhanced CVD release layer to create an etched film. After placing the coated film on the powered electrode with the planarized surface exposed, the reactor chamber was pumped down to a base pressure of less than 1.3 Pa (1 mTorr). O₂ gas was flowed into the chamber at a rate of 100 SCCM. 13.56 MHz RF power was subsequently coupled into the reactor with an applied power of 7500 W. The planarized film was then carried through the reaction zone at a rate of 10 feet/minute (3 meters/minute), to achieve an exposure time of approximately 30 seconds. At the end of this treatment time, the RF power and the gas supply were stopped, and the chamber was returned to atmospheric pressure. The etch time was sufficient to expose the tops of the posts and oxidize the HMDSO coating (which has a slower rate of etching than the planarizing polymer) to silicon carbon oxide.

### Step 5: Peel

3 mL of the resin of Preparative Example 8 was placed between the etched film and a sheet of MELINEX 454. The resin was spread using a hand-roller and then cured by exposing it to 385 nm light from a UV-LED system (home built) for 30 seconds. The peak irradiance was 12.7 mW/cm2 and the total irradiance was 590 mJ/cm². The cured sample was peeled apart by hand, and this transferred the PVB from the etched film to the MELINEX sheet. The result is a nano-structured film consisting of posts with a silicon carbon oxide coating on the post tops. The interstitial space between the post tops has a methylated surface.

### EXAMPLES

### Example 1

Functionalization of Nano-Structured Film with Photoiniferter Silane PE1-B

A 20-mL glass vial was charged with the photoiniferter silane PE1-B (0.20 g), absolute ethanol (9.26 g), water (0.50 g), and acetic acid (0.04 g). The solution was mixed by hand until homogenous. 2 cm by 4 cm cutouts of nano-structured film (from Preparative Example 9) were placed into a dish and immersed in the PE1-B solution. Films were left soaking for 1 hour, then removed and rinsed with excess absolute ethanol. Films were then placed in an oven held at 70° C. for 30 minutes.

### Example 2

Functionalization of Nano-Structured Film with Photoiniferter Silane PE2

Nano-structured film was treated with a solution of PE2 by a method identical to that of Example 1, substituting PE2 for PE1-B.

### Example 3

Functionalization of Nano-Structured Film with Photoiniferter Silane PE3

Nano-structured film was treated with a solution of PE3 by a method identical to that of Example 1, substituting PE3 for PE1-B.

### Example 4

### Growth of Amine Acrylamide Brush Polymer on Nano-Structured Film

The components shown in Table 2 were added to an 8-mL vial. The vial was capped with a rubber septum, then nitrogen gas was bubbled through the solution via a needle inserted through the septum for 10 minutes. A small aliquot of solution was withdrawn by syringe, then an 80 microliter (μL) drop was placed on a piece of glass. The film of Example 1 was placed against the drop (nano-structured side against the drop), so that the solution wetted across the film. A hand-held UV lamp (Model UVGL-58 MINERALIGHT lamp, UVP Inc., Upland, CA) was placed 2 cm above the film, and the film was illuminated with 366 nm UV light for 30 minutes. The film was then soaked in deionized water overnight and air dried. The term “amine acylamide brush polymer” refers to the copolymer formed from acrylamide and N-(3-aminopropyl) methacrylamide.

TABLE 2
Reagent list for Example 4

| COMPONENT | CONCENTRATION | EQUIVALENTS | MILLIMOLES | MASS, g |
|---|---|---|---|---|
| Acrylamide | | 1000 | 2.81 | 0.200 |
| N-(3-aminopropyl) | | 430 | 1.21 | 0.200 |
| methacrylamide | | | | |
| hydrochloride | | | | |
| PE4 (Photoiniferter) | 1% by weight in DMF | 1 | 0.00281 | 0.0586 |
| Water | | | | 1.2 |

### Example 5

### Growth of Amine Acrylamide Brush Polymer on Nano-Structured Film

The brush polymer was prepared using the method described in Example 4, except that the film of Example 2 (functionalized with photoiniferter silane PE2) was used in place of the film of Example 1.

### Example 6

### Growth of Amine Acrylamide Brush Polymer on Nano-Structured Film

The brush polymer was prepared using the method described in Example 4, except that the film of Example 3 (functionalized with photoiniferter silane PE3) was used in place of the film of Example 1.

### Example 7

Covalent Attachment of Fluorescent Dye to Nano-Structured Film with Brush-Coated Tops

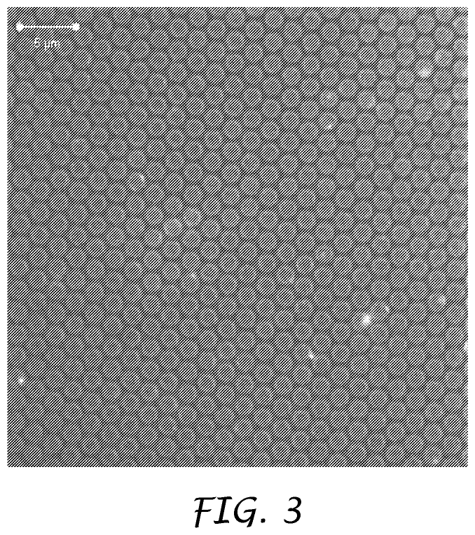

The film of Example 4 was placed in a 12-well plate and rinsed with TE buffer pH 8.0 (2 mL) three times. Approximately 50 μL of a 0.1 mg/mL AF488 NHS ester in TE buffer pH 8.0 was pipetted onto the surface of the nano-structured film functionalized with amine acrylamide brush polymer. After 1 hour, the sample was rinsed with deionized water, dried with nitrogen, and imaged using a confocal microscope according to test method described above. A representative confocal image is shown in FIG. 3. Fluorescence on the post tops demonstrates that brush polymer containing amine functional groups was grown selectively on the posts and not in the interstitial spaces.

### Example 8

Covalent Attachment of Fluorescent Dye to Nano-Structured Film with Brush-Coated Tops

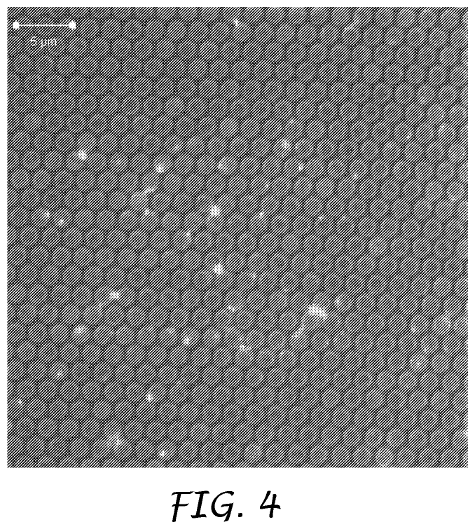

The fluorescent AF488 NHS ester dye was covalently attached to the nano-structured film with amine-containing brush polymer using the same method described for Example 7 but substituting the film of Example 5 (derived from photoiniferter silane PE2) for the film of Example 4. The presence of the dye was confirmed by confocal microscopy according to the test method described above. A representative confocal image is shown in FIG. 4. Fluorescence on the post tops demonstrates that brush polymer containing amine functional groups was grown selectively on the posts and not in the interstitial spaces.

### Example 9

Covalent Attachment of Fluorescent Dye to Nano-Structured Film with Brush-Coated Tops

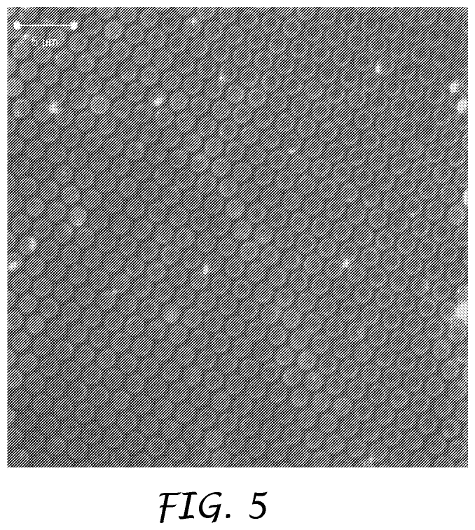

The fluorescent AF488 NHS ester dye was covalently attached to the nano-structured film with amine-containing brush polymer using the same method described for Example 7, substituting the film of Example 6 (derived from photoiniferter silane PE3) for the film of Example 4. The presence of the dye was confirmed by confocal microscopy according to the test method described above. A representative confocal image is shown in FIG. 5. Fluorescence on the post tops demonstrates that brush polymer containing amine functional groups was grown selectively on the posts and not in the interstitial spaces.

### Example 10

### Growth of Azide-Acrylamide Brush Polymer on Nano-Structured Film

The reagents shown in Table 3 were added to a 20 mL vial. The vial was capped with a rubber septum, then nitrogen gas was bubbled through the solution via a needle inserted through the septum for 10 minutes. A small aliquot of solution was withdrawn by syringe, then a 10 microliter drop was placed on a piece of glass. The film of Example 1 was cut into 1 cm×1 cm pieces, then a piece was placed against the drop (nano-structured side against the drop), so that the solution wetted across the film. A hand-held UV lamp (Model UVGL-58 MINERALIGHT lamp, UVP Inc., Upland, CA) was placed 2 cm above the film, and the film was illuminated with 366 nm UV light for 1 hour. The film was then soaked in deionized water overnight and air dried.

TABLE 3
Reagent list for Example 10

| REAGENT | CONCENTRATION | EQUIVALENTS | MILLIMOLES | MASS, g |
|---|---|---|---|---|
| Acrylamide | | 1000 | 28.1 | 2.00 |
| Azide acrylamide | 50% in DMF | 50 | 1.41 | 1.01 |
| PE4 (Photoiniferter) | 1% in DMF | 1 | 0.0281 | 0.586 |
| Water | | | | 8.00 |

### Example 11

Covalent Attachment of Alkyne Oligonucleotide on Nano-Structured Film with Acrylamide Brush-Coated Tops

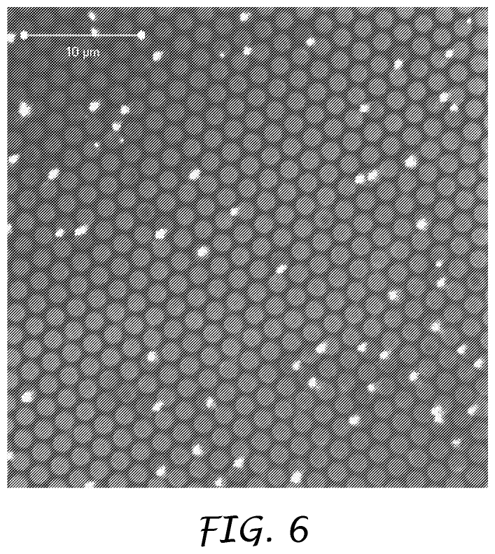

The nano-structured film of Example 10 was functionalized with an alkyne oligonucleotide having a fluorescein group using Cu-catalyzed azide-alkyne cycloaddition. 10 mM potassium phosphate buffer of pH 7.0 (1.429 mL), alkyne oligonucleotide (3 nmol), PMDETA (13.14 microliters), an aqueous solution of copper sulfate pentahydrate (40 mg/mL, 7.49 microliters), and an aqueous solution of sodium ascorbate (400 mg/mL, 6 microliters) were charged into a 1.5 mL DNA LoBind tube sequentially with vortex mixing after addition of each component. The film was placed in an aluminum weighing dish. 500 μL of the above mixture was pipetted onto the surface of the nano-structured film, which was then placed in an oven set at 60° C. for 30 min. The film was taken out and rinsed with deionized water and dried with nitrogen. The presence of the oligonucleotides was confirmed by confocal microscopy. A representative confocal image is shown in FIG. 6. Fluorescence on the post tops demonstrates successful growth of azide-functional brush polymers. The contrast in fluorescence intensity between the post tops and interstitial spaces suggests high spatial selectivity for polymer growth. Some contamination due to free polymer derived from photoinitiator in solution is evident.

### Example 12

Growth of Azide-Acrylamide Brush Polymer on Nano-Structured Film Functionalized with Photoiniferter Silane without Photoiniferter in Solution.

The reagents shown in Table 4 were added to a 20 mL vial. The vial was capped with a rubber septum, then nitrogen gas was bubbled through the solution via a needle inserted through the septum for 10 minutes. A small aliquot of solution was withdrawn by syringe, then a 10 microliter drop was placed on a glass surface. The film of Example 1 was cut into 1 cm×1 cm pieces, then a piece was placed against the drop (nano-structured side against the drop), so that the solution wetted across the film. A hand-held UV lamp (Model UVGL-58 MINERALIGHT lamp, UVP Inc., Upland, CA) was placed 2 cm above the film, and the film was illuminated with 366 nm UV light for 1 hour. The film was then soaked in deionized water overnight and air dried.

TABLE 4
Reagent list for Example 12

| | | | | MASS, |
|---|---|---|---|---|
| REAGENT | CONCENTRATION | EQUIVALENTS | MILLIMOLES | g |
| Acrylamide | | 1000 | 28.1 | 2.00 |
| Azide acrylamide | 50% in DMF | 50 | 1.41 | 1.01 |
| Water | | | | 8.00 |

### Example 13

Covalent Attachment of Alkyne Oligonucleotide on Nano-Structured Film with Azide-Acrylamide Brush-Coated Tops, Previously Functionalized with Photoiniferter Silane and without Photoiniferter in Solution

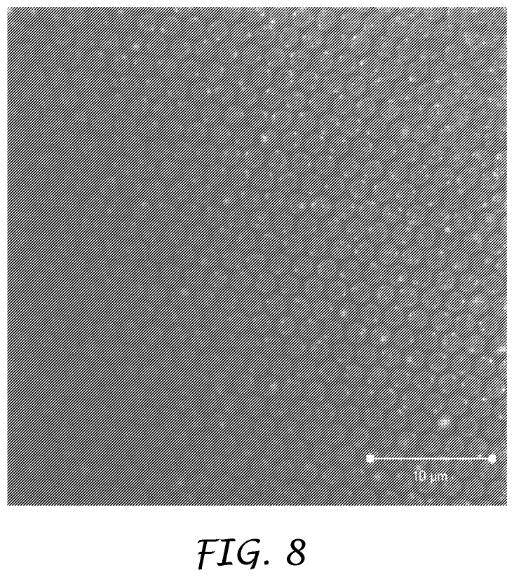

The nano-structured film of Example 12 was functionalized with the alkyne oligonucleotide having a fluorescein group using the same procedure as described in Example 11. A representative confocal image is shown in FIG. 8. Fluorescence on the post tops demonstrates successful growth of azide-functional brush polymers. The low fluorescence intensity indicates less polymer growth in comparison to Example 11. However, the lack of photoiniferter in solution prevents contamination from unbound polymer.

### COMPARATIVE EXAMPLES

### Comparative Example 1

Functionalization of Nano-Structured Film with Acrylamide Silane A 20 mL glass vial was charged with acrylamide silane (0.20 g), absolute ethanol (9.26 g), water (0.50 g), and acetic acid (0.04 g). The solution was mixed by hand until homogenous. 1 cm×1 cm cutouts of nano-structured film were placed into a well plate and silane solution was added by pipette to cover the film pieces. Films were left soaking for 1 hour, then removed and rinsed with excess absolute ethanol. Films were then placed in an oven held at 70° C. for 30 minutes.

### Comparative Example 2

Growth of Azide-Acrylamide Brush Polymer on Nano-Structured Film Functionalized with Acrylamide Silane

The nano-structured film of Comparative Example 1 was subjected to acrylamide polymerization solution using the same procedure as described for Example 10.

### Comparative Example 3

Covalent Attachment of Alkyne Oligonucleotide on Nano-Structured Film with Acrylamide Brush-Coated Tops, Previously Functionalized with Acrylamide Silane and with Photoiniferter in Solution

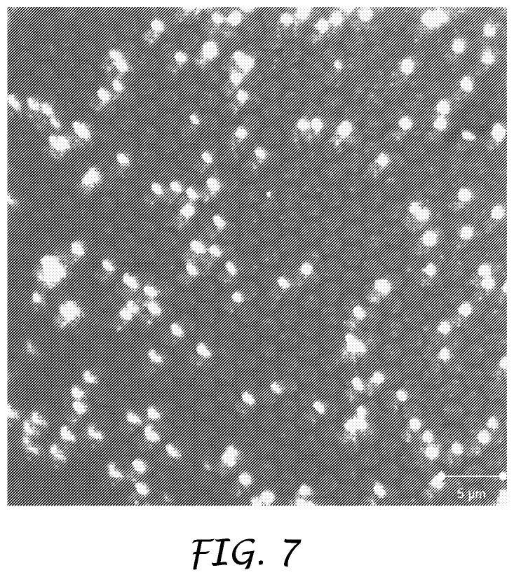

The nano-structured film of Comparative Example 2 was functionalized with alkyne oligonucleotide having a fluorescein group using the same procedure as described in Example 11. A representative confocal image is shown in FIG. 7. Minimal fluorescence on the post tops and a high amount of fluorescence from contaminant suggests that most of the polymer formed is not surface-bound.

1. A first article comprising:
a) a polymeric film;
b) an inorganic oxide coating on at least a first portion of a first surface of the polymeric film, wherein the inorganic oxide coating has a thickness of at least 1 nm and up to 500 nm; and
c) a plurality of compounds of Formula (I) covalently bonded to the inorganic oxide coating through a condensation reaction with a silyl group —Si(R¹)ₙ(R²)₃₋ₙ
Z—(C═S)—S-Q¹-(L)ₚ-[Si(R¹)ₙ(R²)₃₋ₙ]ₘ (I)
wherein
R¹ is an alkyl;
R² is an alkoxy, halo, or a group of formula —O—(C═O)—R³ where R³ is an alkyl;
n is an integer equal to 1, 2, or 3;
m is an integer equal to 1 or 2;
p is an integer equal to 0 or 1;
Z is equal to —N(R⁴)₂, —S—R⁵, —NR⁶R⁷, —OR⁸, or —R⁹ where
R⁴ is an alkyl, aryl, or two R⁴ groups combine to form a 5 or 6 membered ring with nitrogen being one of the ring members and with the ring being saturated or unsaturated;
R⁵ is an alkyl or aryl;
R⁶ is an alkyl;
R⁷ is a nitrogen-containing heteroaryl with 1 or 2 nitrogen ring members;
R⁸ is an alkyl or aryl; and
R⁹ is an aryl;
Q¹ is equal to —CR¹⁰R¹¹—(C═O)—NH—, —CR¹²R¹³—(C═O)—O—, —C(R¹⁴)(C≡N)—, or —CHR¹⁵—R¹⁶— where
R¹⁰ is an alkyl;
R¹¹ is hydrogen, alkyl, or aryl;
R¹² is an alkyl;
R¹³ is hydrogen, alkyl, or aryl;
R¹⁴ is an alkyl;
R¹⁵ is hydrogen or alkyl; and
R¹⁶ is an arylene; and
L is equal to an alkylene, —R¹⁷—O—(C═O)—NH—R¹⁷—, —R¹⁷—(C═O)—NH—R¹⁷—, or —R¹⁷—S—R¹⁷—, or —N[(R¹⁸)—]₂ where
each R¹⁷ is independently an alkylene; and
R¹⁸ is an alkylene.

2. The first article of claim 1, wherein the polymeric film further comprises an anti-biofouling coating on a second portion of the first surface of the polymeric film, wherein the first portion of the first surface is different than the second portion of the first surface.

3. The first article of claim 1, wherein the polymeric film has an array of structures with posts and wells, the inorganic oxide coating being either on a top surface of the posts or on a bottom surface of the wells.

4. The first article of claim 3, wherein the array of structures further comprises an anti-biofouling coating on surfaces of the array of structures where the inorganic oxide coating is not present.

5. The first article of claim 1, wherein the inorganic oxide layer comprises silicon dioxide, silicon carbon oxides, silicon aluminum oxide, titanium oxide, aluminum oxide, tin oxide, germanium oxide, gallium oxide, zinc oxide, indium oxide, or combinations thereof.

6. The first article of claim 1, wherein the anti-biofouling layer comprises a fluoropolymer, a noble metal, or a material with a hydrocarbon surface.

7. The first article of claim 1, further comprising a support film adhered to a second surface of the polymeric film opposite the first surface of the polymeric film.

8. The first article of claim 1, wherein the compound of Formula (I) is selected from 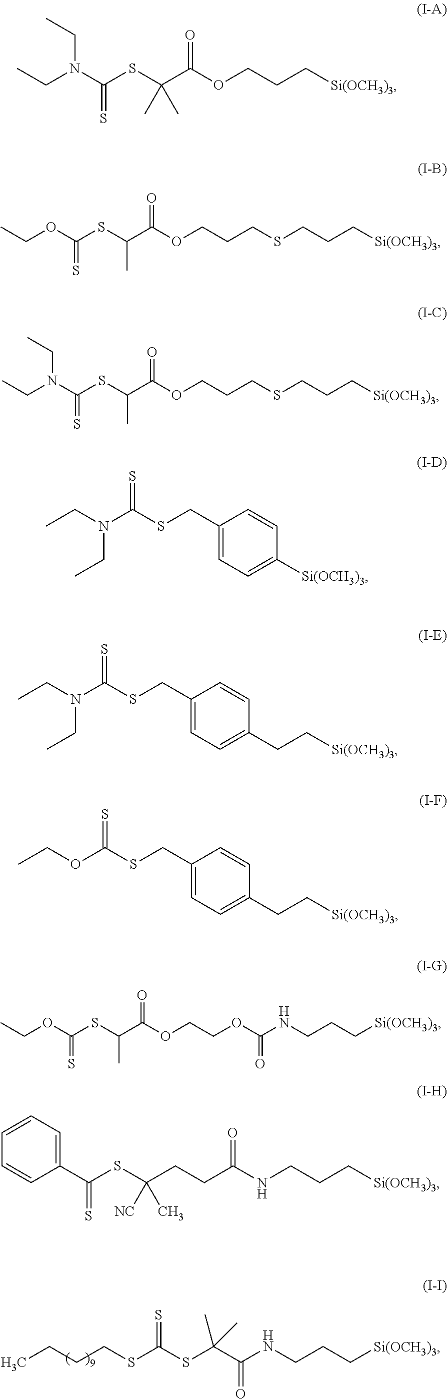
or a mixture thereof.

9. A second article comprising:
a) a polymeric film;
b) an inorganic oxide coating on an outer surface of at least a portion of a first surface of the polymeric film, wherein the inorganic oxide coating has a thickness of at least 1 nm and up to 500 nm;
c) a plurality of polymeric groups of Formula (II) covalently bonded to the metal oxide coating through a condensation reaction with a silyl group (—Si(R¹)ₙ(R²)₃₋ₙ).
Z—(C═S)—S—(POLY)-Q¹-(L)ₚ-[Si(R¹)ₙ(R²)₃₋ₙ]ₘ (II)
wherein
R¹ is an alkyl;
R² is an alkoxy, halo, or a group of formula —O—(C═O)—R³ where R³ is an alkyl;
n is an integer equal to 1, 2, or 3;
m is an integer equal to 1 or 2;
p is an integer equal to 0 or 1;
Z is equal to —N(R⁴)₂, —S—R⁵, —NR⁶R⁷, —OR⁸, or —R⁹ where
R⁴ is an alkyl, aryl, or two R⁴ groups combine to form a 5 or 6 membered ring with nitrogen being one of the ring members and with the ring being saturated or unsaturated;
R⁵ is an alkyl or aryl;
R⁶ is an alkyl;
R⁷ is a nitrogen-containing heteroaryl with 1 or 2 nitrogen ring members;
R⁸ is an alkyl or aryl; and
R⁹ is an aryl;
POLY is a radically polymerized product of at least one monomer having an ethylenically
unsaturated group;
Q¹ is equal to —CR¹⁰R¹¹—(C═O)—NH—, —CR¹²R¹³—(C═O)—O—, —C(R¹⁴)(C≡N)—, or —CHR¹⁵—R¹⁶— where
R¹⁰ is an alkyl;
R¹¹ is hydrogen, alkyl, or aryl;
R¹² is an alkyl;
R¹³ is hydrogen, alkyl, or aryl;
R¹⁴ is an alkyl;
R¹⁵ is hydrogen or alkyl; and
R¹⁶ is an arylene; and
L is equal to an alkylene, —R¹⁷—O—(C═O)—NH—R¹⁷—, —R¹⁷—(C═O)—NH—R¹⁷—, or —R¹⁷—S—R¹⁷—, or —N[(R¹⁸)—]₂ where
each R¹⁷ is independently an alkylene; and
R¹⁸ is an alkylene.

10. The second article of claim 9, wherein POLY is the radically polymerized product of a monomer composition comprising an azido-containing monomer or an amino-containing monomer.

11. The second article of claim 10, wherein the azido-containing monomer is of Formula (III)
CH₂=CR²¹—(C═O)—X¹—[R²²-Q¹⁰]_{y}—R²³ (III)
wherein
R²¹ is hydrogen or methyl;
X¹ is —NH— or —O—;
R²² is an alkylene or a heteroalkylene having at least one oxygen heteroatom;
Q¹⁰ is —(C═O)—X²— or —NH—(C═O)—X²—;
X² is —NH— or —O—;
y is equal to 0 or 1; and
R²³ is either (a) an ether or polyether group terminated with an azido group or (b) a branched hetero-hydrocarbon group terminated with two or more azido groups, the branched hetero-hydrocarbon group having a branching location and comprising (i) an ether or polyether group before the branching location and (ii) an ether or polyether group in each branch after the branching location.

12. A device comprising the second article of claim 9, wherein the device performs a biochemical and/or chemical assay.

13. The device of claim 12, wherein the device determines a nucleic acid sequence of a sample.
